(19) United States
(12) Patent Application Publication
(10) Pub. No.: US 2026/0263588 A1
(43) Pub. Date: Sep. 10, 2026

(54) REAGENTS AND METHODS FOR PREVENTING, TREATING OR LIMITING SEVERE ACUTE RESPIRATORY SYNDROME (SARS) CORONAVIRUS INFECTION

(71) Applicant: PHYLEX BIOSCIENCES, INC., Del Mar, CA (US)

(72) Inventor: Pascal Brandys, Del Mar, CA (US)

(21) Appl. No.: 19/164,759

(22) PCT Filed: May 16, 2024
(86) PCT No.: PCT/US2024/029726

§ 371 Date: Sep. 12, 2025

Related U.S. Application Data
(60) Provisional application No. 63/503,321, filed on May 19, 2023.
(60) Provisional application No. 63/504,955, filed on May 30, 2023.

(51) Int. Cl. A61K 39/215; A61P 37/04; C07K 14/005; G01N 33/569

(52) U.S. Cl. CPC A61K 39/215; A61P 37/04; C07K 14/005; G01N 33/56983; C07K 2318/10; C12N 2770/20022; C12N 2770/20034; G01N 2333/165; G01N 2469/20

(57) ABSTRACT

Polypeptides and nucleic acids encoding the polypeptides are provided that include severe acute respiratory syndrome Co-V-2 (SARS-CoV-2) spike polypeptide receptor-binding domain (RBD) polypeptides that include at least one non-naturally occurring N-linked glycosylation motif in any of the residues 369-372, 388-390, 405-407, 417-419, 437-441, 477-479, 487-489 and 501-503, which are capable of multimerization and thus presenting multiple copies of the RBD to enhance the immune response generated when the polypeptide is administered to a subject.

### SEQUENCE LISTING STATEMENT

A computer readable form of the Sequence Listing is filed with this application by electronic submission and is incorporated into this application by reference in its entirety. The Sequence Listing is contained in the file created on May 6, 2024 having the file name “23-0778-WO.xml” and is 194,211 bytes in size.

### BACKGROUND

A novel zoonotic betacoronavirus that emerged in Wuhan, China at the end of 2019, subsequently named SARS-CoV-2 by the International Committee on Taxonomy of Viruses (ICTV) in January 2020, has resulted in the Coronavirus Disease 2019 (COVID-19) pandemic with a cumulative total of 764 million reported cases and 7 million reported deaths globally as of Apr. 30, 2023.

mRNA-based vaccines for SARS-CoV-2 developed during 2020 have proven to be quite effective in preventing severe COVID-19. However, starting from the third quarter of 2020 new SARS-CoV-2 variants have repeatedly appeared and spread worldwide. Rapidly waning immunity of mRNA vaccines against the delta variant prompted health authorities to recommend or mandate a third injection of legacy mRNA vaccines, with the first U.S. recommendation for a third dose of mRNA vaccine issued on Aug. 12, 2021. Subsequently the omicron variant (B.1.1.529) emerged and became dominant worldwide in less than 2 months from November 2021 to January 2022. In September 2022 the FDA authorized bivalent boosters containing two mRNA components, one of the original strain and the other of the BA.1 or BA.5 omicron variants. However, by the time these boosters were available new post-BA.5 variants had replaced the BA.5 lineage, and subsequently the BQ.1.1, XBB.1 and XBB.1.5 variants each dominated for approximately 3 months. As of Apr. 30, 2023 the latest variant XBB.1.16 is replacing XBB.1.5 worldwide. These new variants are consistently evading immunity against the previous variant and are also consistently equally or more infectious. Currently there is no mRNA vaccine strategy to keep up with the pace of evolution of the SARS-CoV-2 virus.

SARS-CoV-2 is a single, non-segment and positive-stranded RNA virus with envelope. Its genomic RNA consists of 29,903 nucleotides, two thirds of its 5′-encoding nonstructural RNA replicase polyprotein and one third of its 3′-encoding structural proteins, including spike(S), envelope (E), membrane (M), and nucleocapsid (N) proteins.

The SARS-CoV-2 S protein is a type I transmembrane envelope glycoprotein and consists of S1 surface subunit, which is responsible for receptor binding, and S2 transmembrane subunit, which mediates membrane fusion.

The S protein mediates viral entry into host cells by first binding to a host receptor through the receptor-binding domain (RBD) in the S1 subunit and then fusing the viral and host membranes through the S2 subunit. The entry of SARS-CoV-2 is initiated by binding of the S protein to the cellular receptor angiotensin-converting enzyme 2 (ACE2).

In SARS-CoV-2 a fragment of 194 residues spanning the residues 331-524 in S1 subunit is the minimal RBD. Alternatively, a fragment of 204 residues spanning the residues 328-531 in the S1 subunit comprising the minimal RBD is also used in this disclosure. In SARS-CoV-2 the RBD contains a loop region of 72 residues spanning residues 437-508, termed receptor-binding motif (RBM), which makes complete contact with the receptor ACE2.

### DESCRIPTION OF THE FIGURES

FIG. 1. Glycan-masking regions of the SARS-CoV-2 RBD sequence.

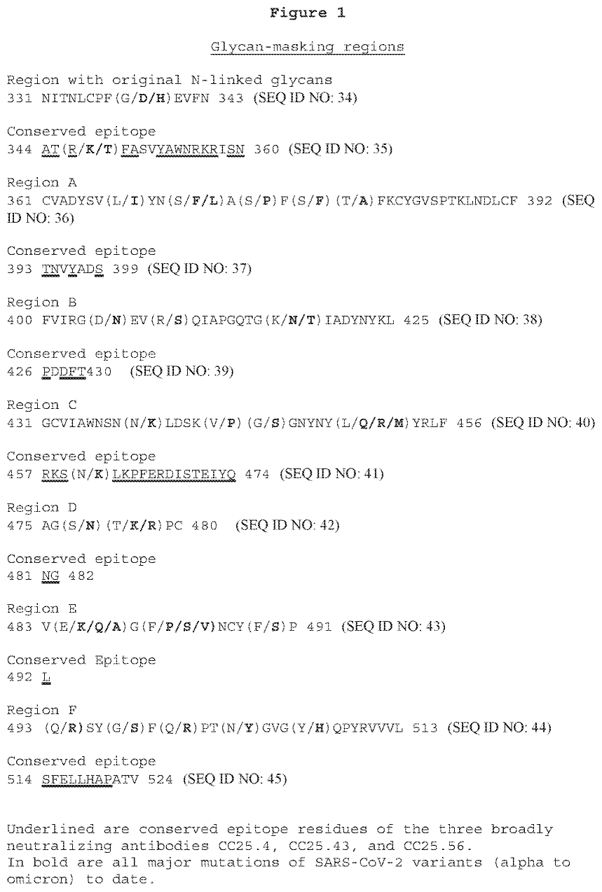

FIG. 2. Designed SARS-CoV-2 RBD mutations generating N-linked glycosylation motifs by region.

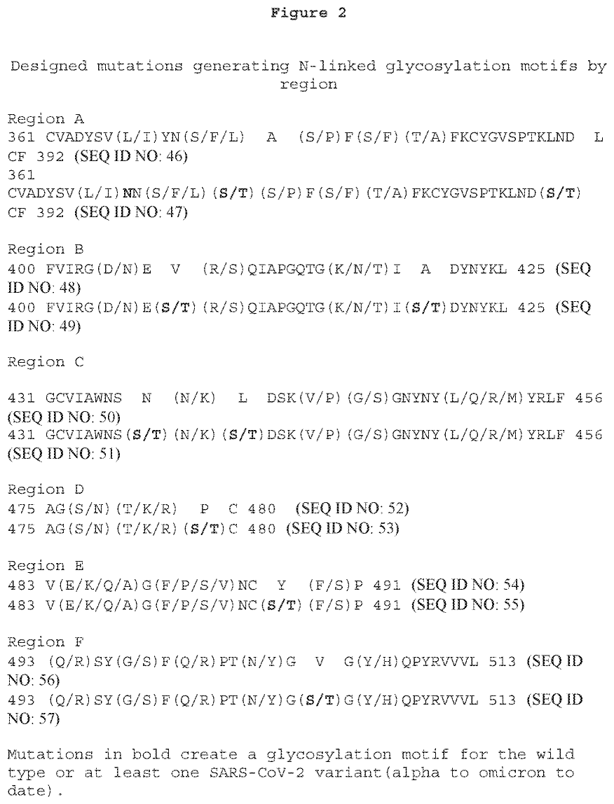

FIG. 3. 3-D structure of N-glycosylated SARS-CoV-2 RBD with designed glycan-masking mutations in Region A.

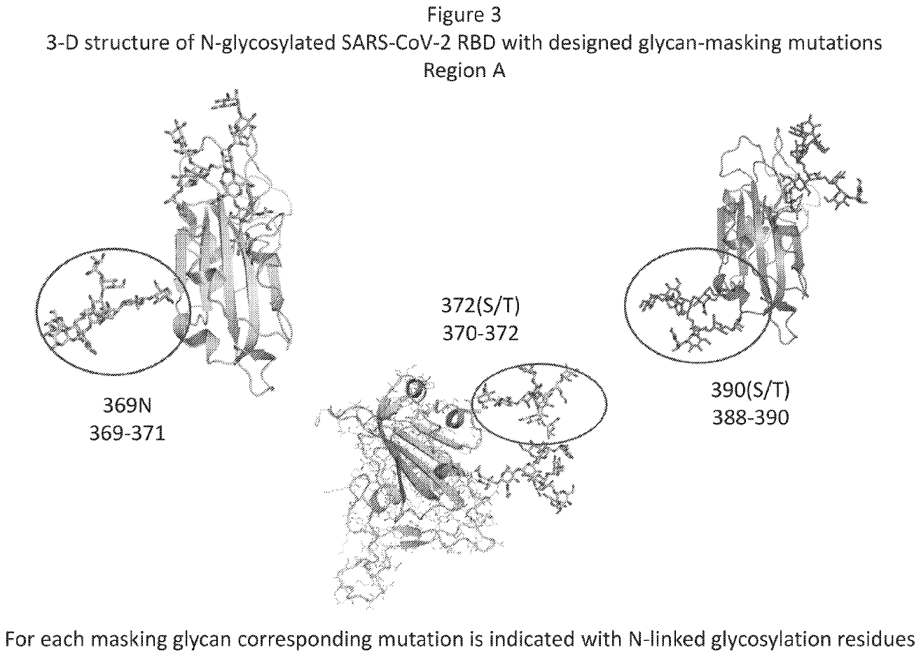

FIG. 4. 3-D structure of N-glycosylated SARS-CoV-2 RBD with designed glycan-masking mutations in Regions B and C.

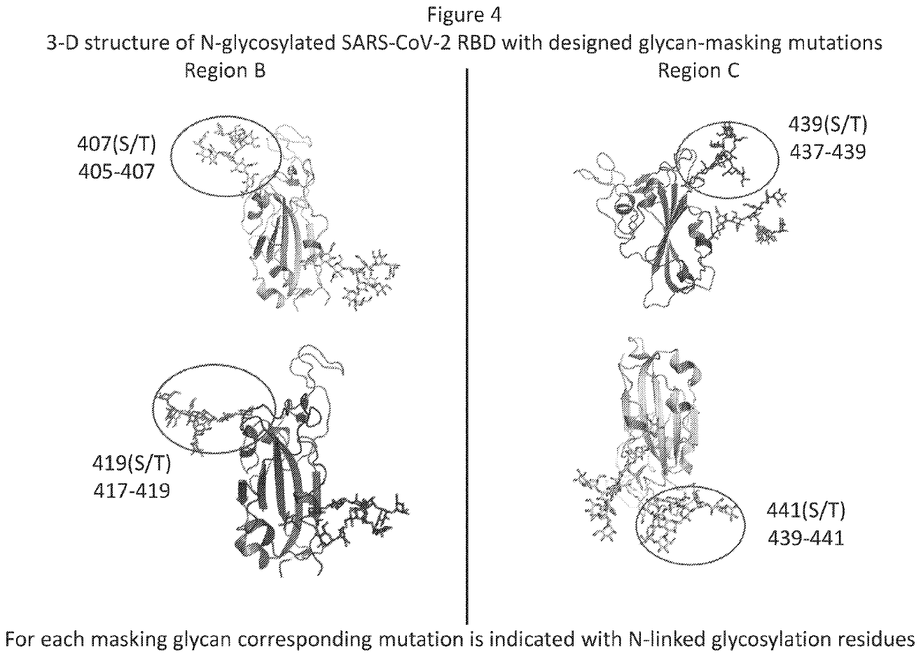

FIG. 5. 3-D structure of N-glycosylated SARS-CoV-2 RBD with designed glycan-masking mutations in Regions D, E and F.

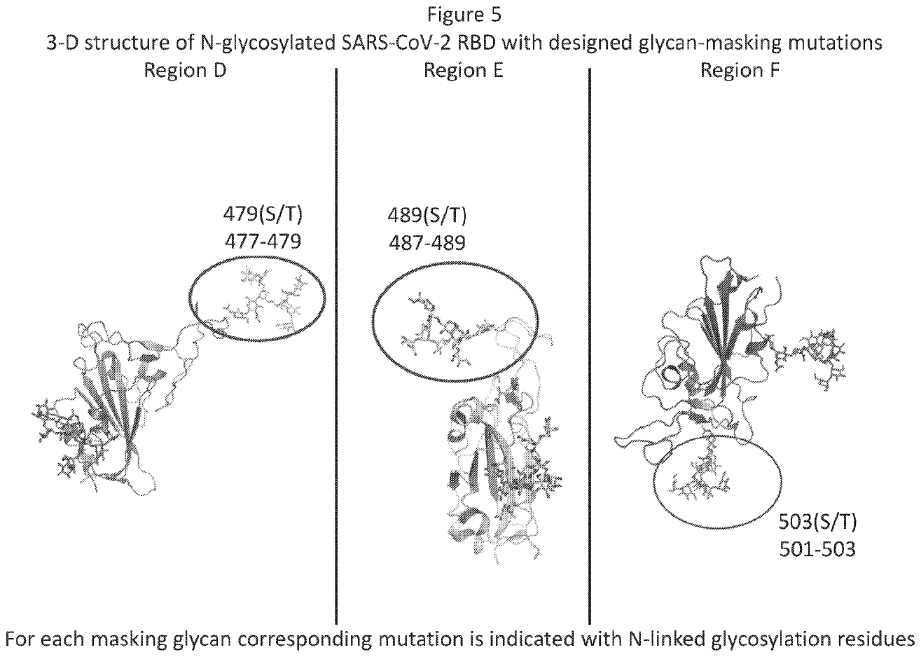

FIG. 6. 3-D structure of N-glycosylated SARS-CoV-2 RBD with 5 designed glycan-masking mutations.

FIG. 7. 3-D structure of N-glycosylated SARS-CoV-2 RBD with 7 designed glycan-masking mutations.

FIG. 8. 3-D structure of N-glycosylated SARS-CoV-2 RBD with 7 designed glycan-masking mutations.

FIG. 9. Optical density results of anti-RBD antibody detection ELISA.

### DETAILED DESCRIPTION

All references cited are herein incorporated by reference in their entirety. As used herein, the singular forms “a”, “an” and “the” include plural referents unless the context clearly dictates otherwise.

All embodiments of any aspect of the disclosure can be used in combination, unless the context clearly dictates otherwise.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used throughout the present application, the terms “protein” or “polypeptide” are used in their broadest sense to refer to a sequence of subunit amino acids. The proteins or polypeptides of the disclosure may comprise L-amino acids, D-amino acids (which are resistant to L-amino acid-specific proteases in vivo), or a combination of D- and L-amino acids. The proteins or polypeptides described herein may be chemically synthesized or recombinantly expressed.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

As used throughout the present application, the term “SARS coronavirus” is used in its broadest sense to designate any highly pathogenic coronavirus phylogenetically related to SARS-COV or SARS-CoV-2.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Parentheses represent variable positions in the polypeptide, with the recited amino acid residues as alternatives in these positions.

An abbreviated amino acid residue preceded or followed by a number indicates the position of the amino acid in a sequence of residues.

### Class 5 Broadly Neutralizing Antibodies Target a Highly Conserved Epitope Region of the RBD

SARS-CoV-2 RBD antibodies have been classified into 5 different classes. Class 1 and 2 antibodies target the RBM, class 3 antibodies (exemplified by the antibody S309, the precursor of Sotrovimab) target a highly conserved epitope outside the RBM, and class 4 antibodies (exemplified by the antibody CR3022) bind to a cryptic epitope outside the RBM. Class 5 antibodies target a site distinct of the epitopes of the class 3 and 4 antibodies, that has been named silent face, lateral site, site V or left flank of the RBD. Site V was identified as the target of the exceptionally cross-reactive pan-sarbecovirus S2H97 RBD antibody exemplifying class 5 antibodies. Antibodies targeting the RBM generally exhibit higher frequency and higher neutralization potency due to direct competition with receptor. Almost all commercially available therapeutic neutralizing antibodies for COVID-19 treatment target the RBM, except Sotrovimab. However, the RBM is highly variable among SARS-CoV-2 variants and all these therapeutic antibodies have now been evaded by at least one SARS-CoV-2 variant. In contrast, the epitope region of three broadly neutralizing class 5 antibodies, CC25.4, CC25.43, and CC25.56, is highly conserved among SARS-CoV-2 variants. S2H97, CC25.4, CC25.56, and CC25.43 are class 5 neutralizing antibodies that target this highly conserved region and retain neutralization activity against SARS-CoV-2 variants. Epitope residues of CC25.4 and CC25.56 are 100% conserved among all SARS-CoV-2 variants to date. Epitope residues of CC25.43 include only one mutation (R436K) in the mu (B.1.621) and omicron BA.1.1 variants and one mutation (R346T) in the omicron BA.2.75.2, BQ.1.1, XBB.1, XBB.1.5, and XBB.1.16 variants.

Rational RBD Immunogen Design with Glycan Masking to Target Desired Conserved Epitopes.

We hypothesized that glycan-masking the vaccine antigen by mutating undesired antigenic sites with an additional N-linked glycosylation motif can refocus the B-cell responses to the desired epitopes, without affecting the overall 3-D structure of the antigen. Our approach was as follows: first, the conserved epitope regions of the RBD for key broadly neutralizing antibodies are excluded from the glycan-masking sites. Second, in order to minimize impact on the 3-D protein structure of the RBD, single amino acid mutations to create N-linked glycosylation motif are used. Third, computational analysis with energy function is used to determine the glycosylated RBD 3-D structure with N-linked glycosylation motif candidates across different variants. Fourth, computational 3-D visualization is used to analyze and maximize the steric effects of glycan-masking, for example, the exposed loops or the protruding sites of the exposed loops on the 3-D RBD protein structure are preferred.

This rational design of the SARS-CoV-2 RBD immunogen with glycan masking is important for future SARS-CoV-2 vaccines in order to limit immune evasion and counter immune imprinting. Rational RBD design with glycan-masking is targeting broadly neutralizing antibodies to conserved epitopes and will therefore limit immune evasion from current and future SARS-CoV-2 variants. Immune imprinting is a phenomenon whereby initial exposure to one immunogen effectively primes B cell memory and limits the development of memory B cells and neutralizing antibodies against new variants of the immunogen. A majority of the population in major countries worldwide has been subject to repeated immunization with S antigen of the wild type followed by breakthrough infection. Immune imprinting is a characteristic of post-vaccination infection that mainly recalls cross-reactive memory B cells elicited by the vaccine, but rarely produces B cells specific of the new variant responsible for the breakthrough infection. Immune imprinting with the wild type S antigen has also been described with second generation bivalent vaccines that include the S antigen of the BA.5, as well as with the latest updated vaccines with the S antigen of the XBB.1.5 variant, that also contains ancestral spike epitopes on the N-terminal and receptor binding domains of the XBB.1.5 spike protein. Accordingly, improved boosters are required to expand the limited immune response created by immune imprinting with first and second generation SARS-CoV-2 vaccines. A booster using rational RBD design with glycan-masking will elicit new broadly neutralizing antibodies targeting epitopes different from the RBM, such as conserved epitopes of class 5 broadly neutralizing antibodies among SARS-CoV-2 variants, and expand the development of memory B cells and neutralizing antibodies in the general population.

### Mutations of SARS-CoV-2 RBD Generating N-Linked Glycosylation Motifs Outside Critical Conserved Epitope Residues.

Six glycan-masking regions located outside the conserved epitope residues of three key class 5 broadly neutralizing antibodies were analyzed using the rational immunogen design strategy described above. The Rosetta™ software suite including algorithms for computational modeling and analysis of protein structures and energy function were used to generate the 3-D structure of N-glycosylated RBD variants and a large number of candidates were visualized and analyzed for glycan conformation.

With the above strategy ten N-linked glycosylation sites on the SARS-CoV-2 RBD across all SARS-CoV-2 variants were discovered that will expand the limited immune response created by immune imprinting with first and second generation SARS-CoV-2 vaccines. The corresponding mutations creating N-linked glycosylation motifs in each region comprise the following residues, wherein residues in parentheses separated by a slash are different options at that residue. Residue numbering is based on S1 subunit numbering.

Region A: residues 361-392
369N
372 (S/T)
390 (S/T)
   Region B: residues 400-425
407 (S/T)
419 (S/T)
   Region C: residues 431-456
439 (S/T)
441 (S/T)
   Region D: residues 475-480
479 (S/T)
   Region E: residues 483-491
489 (S/T)
   Region F: residues 493-513
503 (S/T)

In other embodiments, mutations to create N-linked glycosylation motifs comprise:

Region A: residues 361-392
369N
372S
390T
   Region B: residues 400-425
407T
419S
   Region C: residues 431-456
439S
441T
   Region D: residues 475-480
479S
   Region E: residues 483-491
489T
   Region F: residues 493-513
503T

TABLE 1

| Region A | Region B | Region C | Region D | Region E | Region F |
|---|---|---|---|---|---|
| 369N | 407S | 439S | 479S | 489S | 503S |
| 372S | 407T | 439T | 479T | 489T | 503T |
| 372T | 419S | 441S | | | |
| 390S | 419T | 441T | | | |
| 390T | | | | | |

TABLE 1A
Preferred mutations

| Region A | Region B | Region C | Region D | Region E | Region F |
|---|---|---|---|---|---|
| 369N | 407T | 439S | 479S | 489T | 503T |
| 372S | 419S | 441T | | | |
| 390T | | | | | |

In one embodiment the RBD contains 1, 2, 3, 4, 5, 6, 7 or 8 of the above mutations with no more than two mutations in each of regions A and B, and no more than one mutation in each of regions C, D, E and F

In some embodiments, the SARS-CoV-2 RBD comprises a single mutation of the 10 mutations above. In other embodiments, the SARS-CoV-2 RBD comprises a single mutation in each of regions A, B, C, D, E, and F. In various other non-limiting embodiments, the SARS-CoV-2 RBD comprises a combination of mutations listed in a single row of Table 2 or Table 2A.

TABLE 2

2 mutations in region A
369N, 390(S/T)
372(S/T), 390(S/T)
1 or 2 mutations in region A, and 1 or 2 mutations in region B
369N, 407(S/T)
369N, 419(S/T)
372(S/T), 407(S/T)
372(S/T), 419(S/T)
390(S/T), 407(S/T)
390(S/T), 419(S/T)
369N, 390(S/T), 407(S/T)
369N, 390(S/T), 419(S/T)
372(S/T), 390(S/T), 407(S/T)
372(S/T), 390(S/T), 419(S/T)
369N, 390(S/T), 407(S/T), 419(S/T)
372(S/T), 390(S/T), 407(S/T), 419(S/T)
2 mutations in region B
407(S/T), 419(S/T)
1 mutation in regions A, B, and C
369N, 407(S/T), 439(S/T)
369N, 407(S/T), 441(S/T)
369N, 419(S/T), 439(S/T)
369N, 419(S/T), 441(S/T)
372(S/T), 407(S/T), 439(S/T)
372(S/T), 407(S/T), 441(S/T)
372(S/T), 419(S/T), 439(S/T)
372(S/T), 419(S/T), 441(S/T)
390(S/T), 407(S/T), 439(S/T)
390(S/T), 407(S/T), 441(S/T)
390(S/T), 419(S/T), 439(S/T)
390(S/T), 419(S/T), 441(S/T)
1 or 2 mutations in region A and 1 mutation in region C
369N, 439(S/T)
369N, 441(S/T)
372(S/T), 439(S/T)
372(S/T), 441(S/T)
390(S/T), 439(S/T)
390(S/T), 441(S/T)
369N, 390(S/T), 439(S/T)
369N, 390(S/T), 441(S/T)
372(S/T), 390(S/T), 439(S/T)
372(S/T), 390(S/T), 441(S/T)
1 or 2 mutations in region B and 1 mutation in region C
407(S/T), 439(S/T)
407(S/T), 441(S/T)
419(S/T), 439(S/T)
419(S/T), 441(S/T)
407(S/T), 419(S/T), 439(S/T)
407(S/T), 419(S/T), 441(S/T)
1 or 2 mutations in region A; 1 or 2 mutations in region B, and 1
mutation in region C
369N, 407(S/T), 439(S/T)
369N, 407(S/T), 441(S/T)
369N, 419(S/T), 439(S/T)
369N, 419(S/T) 441(S/T)
372(S/T), 407(S/T), 439(S/T)
372(S/T), 407(S/T), 441(S/T)
372(S/T), 419(S/T), 439(S/T)
372(S/T), 419(S/T), 441(S/T)
390(S/T), 407(S/T), 439(S/T)
390(S/T), 407(S/T), 441(S/T)
390(S/T), 419(S/T), 439(S/T)
390(S/T), 419(S/T), 441(S/T)
369N, 390(S/T), 407(S/T), 439(S/T)
369N, 390(S/T), 407(S/T), 441(S/T)
369N, 390(S/T), 419(S/T), 439(S/T)
369N, 390(S/T), 419(S/T), 441(S/T)
372(S/T), 390(S/T), 407(S/T), 439(S/T)
372(S/T), 390(S/T), 407(S/T), 441(S/T)
372(S/T), 390(S/T), 419(S/T), 439(S/T)
372(S/T), 390(S/T), 419(S/T), 441(S/T)
369N, 390(S/T), 407(S/T), 419(S/T), 439(S/T)
369N, 390(S/T), 407(S/T), 419(S/T), 441(S/T)
372(S/T), 390(S/T), 407(S/T), 419(S/T), 439(S/T)
372(S/T), 390(S/T), 407(S/T), 419(S/T), 441(S/T)

TABLE 2A

2 mutations in region A
369N, 390T
372S, 390T
1 or 2 mutations in region A, and 1 or 2 mutations in region B
369N, 407T
369N, 419S
372S, 407T
372S, 419S
390T, 407T
390T, 419S
369N, 390T, 407T
369N, 390T, 419S
372S, 390T, 407T
372S, 390T, 419S
369N, 390T, 407T, 419S
372S, 390T, 407T, 419S
2 mutations in region B
407T, 419S
1 mutation in regions A, B, and C
369N, 407T, 439S
369N, 407T, 441T
369N, 419S, 439S
369N, 419S, 441T
372S, 407T, 439S
372S, 407T, 441T
372S, 419S, 439S
372S, 419S, 441T
390T, 407T, 439S
390T, 407T, 441T
390T, 419S, 439S
390T, 419S, 441T
1 or 2 mutations in region A and 1 mutation in region C
369N, 439S
369N, 441T
372S, 439S
372S, 441T
390T, 439S
390T, 441T
369N, 390T, 439S
369N, 390T, 441T
372S, 390T, 439S
372S, 390T, 441T
1 or 2 mutations in region B and 1 mutation in region C
407T, 439S
407T, 441T
419S, 439S
419S, 441T
407T, 419S, 439S
407T, 419S, 441T
1 or 2 mutations in region A; 1 or 2 mutations in region B, and 1
mutation in region C
369N, 407T, 439S
369N, 407T, 441T
369N, 419S, 439S
369N, 419S 441T
372S, 407T, 439S
372S, 407T, 441T
372S, 419S, 439S
372S, 419S, 441T
390T, 407T, 439S
390T, 407T, 441T
390T, 419S, 439S
390T, 419S, 441T
369N, 390T, 407T, 439S
369N, 390T, 407T, 441T
369N, 390T, 419S, 439S
369N, 390T, 419S, 441T
372S, 390T, 407T, 439S
372S, 390T, 407T, 441T
372S, 390T, 419S, 439S
372S, 390T, 419S, 441T
369N, 390T, 407T, 419S, 439S
369N, 390T, 407T, 419S, 441T
372S, 390T, 407T, 419S, 439S
372S, 390T, 407T, 419S, 441T

In any embodiment of the combinations of mutations shown in Table 2, the mutations may further comprise 479 (S/T). In any embodiment of the combinations of mutations shown in Table 2A, the mutations may further comprise 479S. In a further embodiment of the combination of mutations shown in Table 2, the mutations may further comprise 489 (S/T). In a further embodiment of the combination of mutations shown in Table 2A, the mutations may further comprise 489T. In another embodiment of the combination of mutations shown in Table 2, the mutations may further comprise 503 (S/T). In another embodiment of the combination of mutations shown in Table 2A, the mutations may further comprise 503T. In other embodiments, the combinations of mutations shown in Table 2 may further comprise (a) 479 (S/T) and 489 (S/T); (b) 479 (S/T) and 503 (S/T); (c) 489 (S/T) and 503 (S/T); or (d) 479 (S/T), 489 (S/T) and 503 (S/T). In other embodiments the combinations of mutations shown in Table 2A may further comprise (a) 479S and 489T; (b) 479S and 503T; (c) 489T and 503T; or (d) 479S, 489T and 503T.

In another embodiment, the SARS-CoV-2 RBD comprises a combination of mutations selected from the group consisting of (a) 479 (S/T) and 489 (S/T); (b) 479 (S/T) and 503 (S/T); (c) 489 (S/T) and 503 (S/T); or (d) 479 (S/T), 489 (S/T) and 503 (S/T). In one such embodiment, the combination of mutations may further comprise 439 (S/T) or 441 (S/T). In another embodiment, the combination of mutations may further comprise (i) 369N, (ii) 372 (S/T) or 390 (S/T). In another embodiment, the combination of mutations may further comprise 407 (S/T) or 419 (S/T).

In another embodiment, the SARS-CoV-2 RBD comprises a combination of mutations selected from the group consisting of (a) 479S and 489T; (b) 479S and 503T; (c) 489T and 503T; or (d) 479S, 489T and 503T. In one such embodiment, the combination of mutations may further comprise 439S or 441T. In another embodiment, the combination of mutations may further comprise (i) 369N, (ii) 372S or 390T. In another embodiment, the combination of mutations may further comprise 407T or 419S.

The above mutations are included as options in SEQ ID NO: 1 of all SARS-CoV-2 RBD variants (alpha to omicron). SEQ IDs NO: 1-6 residue numbering is based on S1 subunit number minus 330. In other words, Residue 331 of S1=Residue 1 of SEQ ID NO: 1-6.

(SEQ ID NO: 1)
NITNLCPF(G/D/H)EVFNAT(R/K/T)FASVYAWNRKRISNCVADYSV
(L/I)(Y/N)N(S/F/L)(A/S/T)(S/P)F(S/F)(T/A)FKCYGVSP
TKLND(L/S/T)CFTNVYADSFVIRG(D/N)E(V/S/T)(R/S)QIAPG
QTG(K/N/T)I(A/S/T)DYNYKLPDDFTGCVIAWNS(N/S/T)(N/K)
(L/S/T)DSK(V/P)(G/S)GNYNY(L/Q/R/M)YRLFRKS(N/K)LKP
FERDISTEIYQAG(S/N)(T/K/R)(P/S/T)CNGV(E/K/Q/A)G(F/
P/S/V)NC(Y/S/T)(F/S)PL(Q/R)SY(G/S)F(Q/R)PT(N/Y)G
(V/S/T)G(Y/H)QPYRVVVLSFELLHAPATV
(all SARS-COV-2 variants RBD glycosylation
motifs)
(SEQ ID NO: 20)
NITNLCPF(G/D/H)EVFNAT(R/K/T)FASVYAWNRKRISNCVADYSV
(L/I)(Y/N)N(S/F/L)(A/S)(S/P)F(S/F)(T/A)FKCYGVSPTK
LND(L/T)CFTNVYADSFVIRG(D/N)E(V/T)(R/S)QIAPGQTG(K/
N/T)I(A/S)DYNYKLPDDFTGCVIAWNS(N/S)(N/K)(L/T)DSK
(V/P)(G/S)GNYNY(L/Q/R/M)YRLFRKS(N/K)LKPFERDISTEIY
QAG(S/N)(T/K/R)(P/S)CNGV(E/K/Q/A)G(F/P/S/V)NC(Y/
T)(F/S)PL(Q/R)SY(G/S)F(Q/R)PT(N/Y)G(V/T)G(Y/H)QPY
RVVVLSFELLHAPATV
(all SARS-COV-2 variants RBD glycosylation motifs
with preferred mutations)

A subset of 13 of the above mutations are applicable to the wild type SARS-CoV-2 RBD sequence:

(SEQ ID NO: 2)
NITNLCPFGEVFNATRFASVYAWNRKRISNCVADYSVL(Y/N)NS(A/
S/T)SFSTFKCYGVSPTKLND(L/S/T)CFTNVYADSFVIRGDEVRQI
APGQTGKIADYNYKLPDDFTGCVIAWNS(N/S/T)N(L/S/T)DSKVG
GNYNYLYRLFRKSNLKPFERDISTEIYQAGSTPCNGVEGFNC(Y/S/
T)FPLQSYGFQPTNG(V/S/T)GYQPYRVVVLSFELLHAPATV
(SARS-COV-2 RBD glycosylation motifs)
(SEQ ID NO: 21)
NITNLCPFGEVFNATRFASVYAWNRKRISNCVADYSVL(Y/N)NS(A/
S)SFSTFKCYGVSPTKLND(L/T)CFTNVYADSFVIRGDEVRQIAPGQ
TGKIADYNYKLPDDFTGCVIAWNS(N/S)N(L/T)DSKVGGNYNYLYR
LFRKSNLKPFERDISTEIYQAGSTPCNGVEGFNC(Y/T)FPLQSYGFQ
PTNG(V/T)GYQPYRVVVLSFELLHAPATV
(SARS-COV-2 RBD glycosylation motifs with
preferred mutations)

Another subset of 16 of the above mutations are applicable to the latest omicron XBB.1.16 variant RBD sequence:

(SEQ ID NO: 3)
NITNLCPFHEVFNATTFASVYAWNRKRISNCVADYSVIYNF(A/S/T)P
FFAFKCYGVSPTKLND(L/S/T)CFTNVYADSFVIRGNE(V/S/T)SQI
APGQTGNI(A/S/T)DYNYKLPDDFTGCVIAWNS(N/S/T)K(L/S/T)
DSKPSGNYNYLYRLFRKSKLKPFERDISTEIYQAGNR(P/S/T)CNGV
AGPNC(Y/S/T)SPLQSYGFRPTYGVGHQPYRVVVLSFELLHAPATV
(SARS-COV-2 XBB.1.16 RBD glycosylation motifs)
(SEQ ID NO: 22)
NITNLCPFHEVFNATTFASVYAWNRKRISNCVADYSVIYNF(A/S)PFF
AFKCYGVSPTKLND(L/T)CFTNVYADSFVIRGNE(V/T)SQIAPGQTG
NI(A/S)DYNYKLPDDFTGCVIAWNS(N/S)K(L/T)DSKPSGNYNYLY
RLFRKSKLKPFERDISTEIYQAGNR(P/S)CNGVAGPNC(Y/T)SPLQS
YGFRPTYGVGHQPYRVVVLSFELLHAPATV
(SARS-COV-2 XBB.1.16 RBD glycosylation motifs
with preferred mutations)

SEQ ID NO: 4 is the reference sequence of all SARS-CoV-2 variants (alpha to omicron) RBD as of Apr. 30, 2023:

(SEQ ID NO: 4)
NITNLCPF(G/D/H)EVFNAT(R/K/T)FASVYAWNRKRISNCVADYSV
(L/I)YN(S/F/L)A(S/P)F(S/F)(T/A)FKCYGVSPTKLNDLCFTN
VYADSFVIRG(D/N)EV(R/S)QIAPGQTG(K/N/T)IADYNYKLPDDF
TGCVIAWNSN(N/K)LDSK(V/P)(G/S)GNYNY(L/Q/R/M)YRLFRK
S(N/K)LKPFERDISTEIYQAG(S/N)(T/K/R)PCNGV(E/K/Q/A)G
(F/P/S/V)NCY(F/S)PL(Q/R)SY(G/S)F(Q/R)PT(N/Y)GVG
(Y/H)QPYRVVVLSFELLHAPATV
(all SARS-COV-2 variants RBD)

SEQ ID NO: 5 is the reference sequence of SARS-CoV-2 wild type RBD:

(SEQ ID NO: 5)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATV
(SARS-COV-2 RBD)

SEQ ID NO: 6 is the reference sequence of SARS-CoV-2 XBB.1.16 omicron RBD:

(SEQ ID NO: 6)
NITNLCPFHEVFNATTFASVYAWNRKRISNCVADYSVIYNFAPFFAFKC
YGVSPTKLNDLCFTNVYADSFVIRGNEVSQIAPGQTGNIADYNYKLPDD
FTGCVIAWNSNKLDSKPSGNYNYLYRLFRKSKLKPFERDISTEIYQAGN
RPCNGVAGPNCYSPLQSYGFRPTYGVGHQPYRVVVLSFELLHAPATV
(SARS-COV-2 XBB.1.16 RBD)

In one aspect, the disclosure provides isolated polypeptide comprising:
(a) a SARS-CoV-2 receptor binding domain (RBD) comprising an amino acid sequence at least 70%, 75%, 80%, 85%, 90%, 91%, 92%, 93%, 94%, 95%, 96%, 97%, 98%, 99%, or 100% identical to the amino acid sequence of any one of SEQ ID NOS: 1-3, and including one or more residue selected from the group consisting of 369N, 372 (S/T), 390 (S/T), 407 (S/T), 419 (S/T), 439 (S/T), 441 (S/T), 479 (S/T), 489 (S/T), and 503 (S/T); and
(b) a multimerization domain.

The polypeptides of the disclosure comprise a multimerization domain. In one embodiment, the multimerization domains of SEQ ID NOS: 27 and 28 can be used to generate multimers comprising 3, 4, 6 or 8 copies of the isolated polypeptides of the disclosure. RMKQIEDKIEEILSKIYHIENEIARIKKLIGER (SEQ ID NO: 27) (Coiled trimerization motif) MKVKQLEDVVEELLSVNYHLENVVARLKKLVGER (SEQ ID NO: 28) (Tetramerization motif having 4 helices curling around each other in helical manner)

In another embodiment the polypeptide is capable of generating multimers comprising at least 60 copies of the isolated polypeptide. For example, the polypeptides can be engineered via genetic fusion to create 60-mer multimers. These constructs may be expressed, for example, in Chinese hamster ovary (CHO) cells and purified using standard nickel and size exclusion methods. By size exclusion chromatography with multi-angle light scattering (SEC-MALS), each construct is shown to have the correct molecular weight according to its intended multimeric state. The antigenic profiles of the constructs are tested and the results show binding to neutralizing antibodies.

The polypeptide is capable of multimerization and thus presenting multiple copies of the RBD to enhance the immune response generated when the polypeptide is administered to a subject. Any multimerization can be used that is capable of generating multimers. In one embodiment, the multimerization domain comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO: 7 or 8.

(SEQ ID NO: 7)
MQIY(E/C)GK(L/C)(T/G)AEGLRFGIVASR(F/A)NHALVDRLVEG
AIDAIV(R/C)(H/F/M)GGREEDITLV(R/C)V(P/C)GSWEIP(V/
C)AAGELARKEDIDAVIAIGVL(I/C)RGA(T/C)(P/G)(H/S)FDYI
ASEVSKGLADLS(L/C)ELRKPITFGVITA(D/C)TLEQAIE(R/A)AG
T(K/C)HGNKGWEAAL(S/C)AIEMANLFKSLR
(Lumazine synthase(LS)variants)
(SEQ ID NO: 8)
MQIYEGKLTAEGLRFGIVASRANHALVDRIVEGAIDAIVRHGGREEDIT
LVRVCGSWEIPVAAGELARKEDIDAVIAIGVLCRGATPSFDYIASEVSK
GLADLSLELRKPITFGVITADTLEQAIEAAGTCHGNKGWEAALCAIEMA
NLFKSLR
(Lumazine synthase(LS))

In this embodiment, the multimerization platform comprises lumazine synthase. The multimerization domains of SEQ ID NOS: 7 and 8 can be used to generate multimers comprising 60 copies of the isolated polypeptides of the disclosure.

In one embodiment where the linker comprises SEQ ID NO: 7, at least 1, 2, 3, 4, 5, 6, 7, 8, 9, 10, 11, 12, 13, 14, 15, 16, 17 or all 18 of the residues bounded by parentheses is the first listed residue.

In another embodiment the polypeptides of the disclosure may further comprise an amino acid linker between the RBD and the multimerization domain. Any amino acid linker may be used as suitable for an intended purpose. In one embodiment, the linker is a Gly-Ser rich linker (i.e.: 50%, 60%, 70%, 80%, 90%, 95%, or 100% made up of Gly or Ser residues). The combination of flexible and hydrophilic residues in these linkers limits the formation of secondary structures and reduces the likelihood that the linkers will interfere with the folding and function of the protein domains. In one specific embodiment, the linker comprises or consists of (GGS)ₙGGG (SEQ ID NO: 33), wherein n is 3-7, 3-6, 3-5, 3-4, 4-7, 4-6, 4-5, 5-7, 5-6, 3, 4, 5, 6, or 7.

The multimerization may be N-terminal or C-terminal to the RBD. In one specific embodiment, the RBD is carboxy-terminal to the multimerization domain.

In another embodiment the N-linked glycans associated with the mutations 479 (S/T) and 489 (S/T) are excluded from the glycosylated RBD.

In other embodiments, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 9-11, wherein n is 3-7, 3-6, 3-5, 3-4, 4-7, 4-6, 4-5, 5-7, 5-6, 3, 4, 5, 6, or 7.

(SEQ ID NO: 9)
MQIY(E/C)GK(L/C)(T/G)AEGLRFGIVASR(F/A)NHALVDRLVEG
AIDAIV(R/C)(H/F/M)GGREEDITLV(R/C)V(P/C)GSWEIP(V/
C)AAGELARKEDIDAVIAIGVL(I/C)RGA(T/C)(P/G)(H/S)FDYI
ASEVSKGLADLS(L/C)ELRKPITFGVITA(D/C)TLEQAIE(R/A)AG
T(K/C)HGNKGWEAAL(S/C)AIEMANLFKSLR(GGS)ₙGGGNITNLCP
F(G/D/H)EVFNAT(R/K/T)FASVYAWNRKRISNCVADYSV(L/I)
(Y/N)N(S/F/L)(A/S/T)(S/P)F(S/F)(T/A)FKCYGVSPTKLND
(L/S/T)CFTNVYADSFVIRG(D/N)E(V/S/T)(R/S)QIAPGQTG
(K/N/T)I(A/S/T)DYNYKLPDDFTGCVIAWNS(N/S/T)(N/K)(L
/S/T)DSK(V/P)(G/S)GNYNY(L/Q/R/M)YRLFRKS(N/K)LKPFE
RDISTEIYQAG(S/N)(T/K/R)(P/S/T)CNGV(E/K/Q/A)G(F/P/
S/V)NC(Y/S/T)(F/S)PL(Q/R)SY(G/S)F(Q/R)PT(N/Y)G(V/
S/T)G(Y/H)QPYRVVVLSFELLHAPATV
(SEQ ID NO: 7 + linker + SEQ ID NO: 1)
(SEQ ID NO: 10)
MQIY(E/C)GK(L/C)(T/G)AEGLRFGIVASR(F/A)NHALVDRLVEG
AIDAIV(R/C)(H/F/M)GGREEDITLV(R/C)V(P/C)GSWEIP(V/
C)AAGELARKEDIDAVIAIGVL(I/C)RGA(T/C)(P/G)(H/S)FDYI
ASEVSKGLADLS(L/C)ELRKPITFGVITA(D/C)TLEQAIE(R/A)AG
T(K/C)HGNKGWEAAL(S/C)AIEMANLFKSLR(GGS)ₙGGGNITNLCP
FGEVFNATRFASVYAWNRKRISNCVADYSVL(Y/N)NS(A/S/T)SFST
FKCYGVSPTKLND(L/S/T)CFTNVYADSFVIRGDEVRQIAPGQTGKIA
DYNYKLPDDFTGCVIAWNS(N/S/T)N(L/S/T)DSKVGGNYNYLYRLF
RKSNLKPFERDISTEIYQAGSTPCNGVEGFNC(Y/S/T)FPLQSYGFQP
TNG(V/S/T)GYQPYRVVVLSFELLHAPATV
(SEQ ID NO: 7 + linker + SEQ ID NO: 2
(SEQ ID NO: 11)
MQIY(E/C)GK(L/C)(T/G)AEGLRFGIVASR(F/A)NHALVDRIVEG
AIDAIV(R/C)(H/F/M)GGREEDITLV(R/C)V(P/C)GSWEIP(V/
C)AAGELARKEDIDAVIAIGVL(I/C)RGA(T/C)(P/G)(H/S)FDYI
ASEVSKGLADLS(L/C)ELRKPITFGVITA(D/C)TLEQAIE(R/A)AG
T(K/C)HGNKGWEAAL(S/C)AIEMANLFKSLR(GGS)ₙGGGNITNLCP
FHEVFNATTFASVYAWNRKRISNCVADYSVIYNF(A/S/T)PFFAFKCY
GVSPTKLND(L/S/T)CFTNVYADSFVIRGNE(V/S/T)SQIAPGQTGN
I(A/S/T)DYNYKLPDDFTGCVIAWNS(N/S/T)K(L/S/T)DSKPSGN
YNYLYRLFRKSKLKPFERDISTEIYQAGNR(P/S/T)CNGVAGPNC(Y/
S/T)SPLQSYGFRPTYGVGHQPYRVVVLSFELLHAPATV
(SEQ ID NO: 7 + linker + SEQ ID NO: 3)

In specific embodiments, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence of any one of SEQ ID NO: 12-14, 17-19, and 23-24.

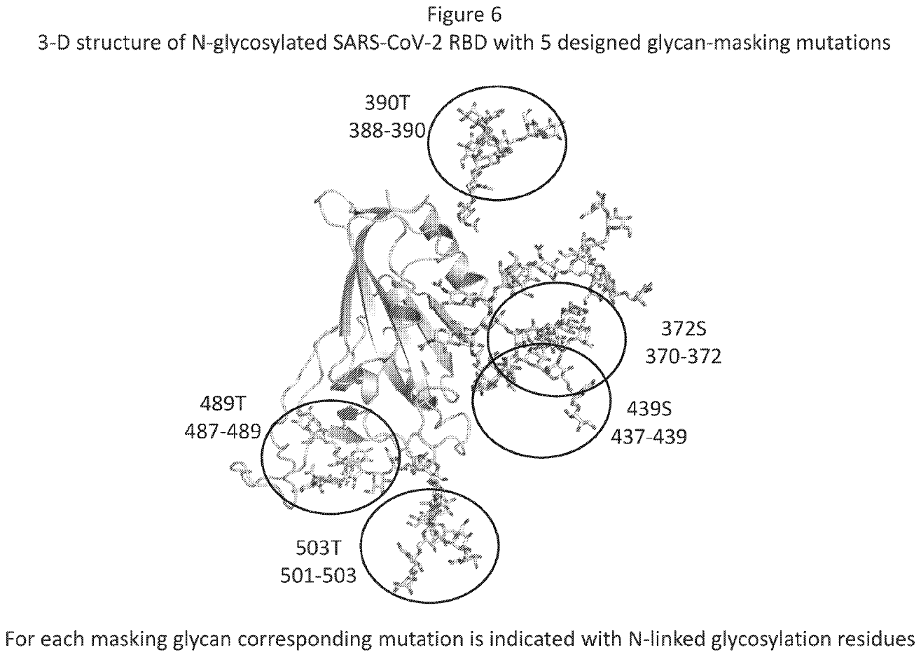

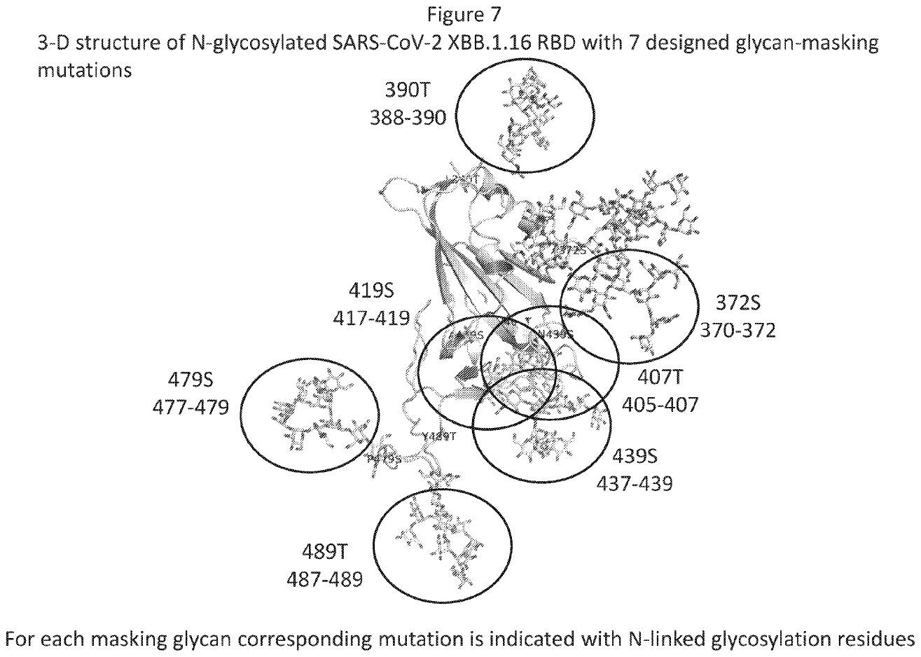

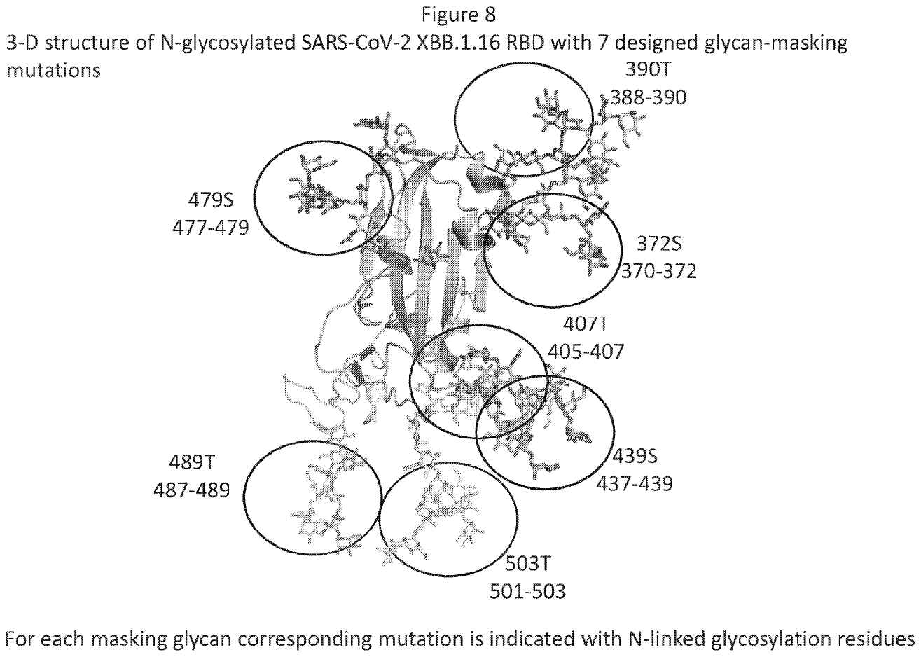

(SEQ ID NO: 12)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPF(G/D/H)
EVFNAT(R/K/T)FASVYAWNRKRISNCVADYSV(L/I)(Y/N)N(S/F/L)(A/S/T)(S/
P)F(S/F)(T/A)FKCYGVSPTKLND(L/S/T)CFTNVYADSFVIRG(D/N)E(V/S/T)
(R/S)QIAPGQTG(K/N/T)I(A/S/T)DYNYKLPDDFTGCVIAWNS(N/S/T)(N/K)(L/S/
T)DSK(V/P)(G/S)GNYNY(L/Q/R/M)YRLFRKS(N/K)LKPFERDISTEIYQAG(S/N)
(T/K/R)(P/S/T)CNGV(E/K/Q/A)G(F/P/S/V)NC(Y/S/T)(F/S)PL(Q/R)SY
(G/S)F(Q/R)PT(N/Y)G(V/S/T)G(Y/H)QPYRVVVLSFELLHAPATVCGPKKST
(SEQ ID NO: 8 + (GGS)₄GGG + RFP + SEQ ID NO: 1 + CGPKKST)
(SEQ ID NO: 13)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVL(Y/N)NS(A/S/T)SFSTFKCYGVSPTKLND(L/S/T)
CFTNVYADSFVIRGDEVRQIAPGQTGKIADYNYKLPDDFTGCVIAWNS(N/S/T)N(L/S/T)
DSKVGGNYNYLYRLFRKSNLKPFERDISTEIYQAGSTPCNGVEGFNC(Y/S/T)FPLQSYG
FQPTNG(V/S/T)GYQPYRVVVLSFELLHAPATVCGPKKST
(SEQ ID NO: 8 +(GGS)₄GGG + RFP + SEQ ID NO: 2 + CGPKKST)
(SEQ ID NO: 14)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFHEVFNA
TTFASVYAWNRKRISNCVADYSVIYNF(A/S/T)PFFAFKCYGVSPTKLND(L/S/T)CFTN
VYADSFVIRGNE(V/S/T)SQIAPGQTGNI(A/S/T)DYNYKLPDDFTGCVIAWNS(N/S/T)
K(L/S/T)DSKPSGNYNYLYRLFRKSKLKPFERDISTEIYQAGNR(P/S/T)CNGVAGPNC
(Y/S/T)SPLQSYGFRPTYGVGHQPYRVVVLSFELLHAPATVCGPKKST
(SEQ ID NO: 8 +(GGS)₄GGG + RFP + SEQ ID NO: 3 + CGPKKST)
(SEQ ID NO: 17)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFGEVFNA
TRFASVYAWNRKRISNCVADYSVLYNSSSESTFKCYGVSPTKLNDTCFTNVYADSFVIRGDE
VRQIAPGQTGKIADYNYKLPDDFTGCVIAWNSSNLDSKVGGNYNYLYRLFRKSNLKPFERDI
STEIYQAGSTPCNGVEGFNCTFPLQSYGFQPTNGTGYQPYRVVVLSFELLHAPATVCGPKKS
T
(Masked SARS-CoV-2 RBD fused with LS i.e. SEQ ID NO: 13 with 5
specific mutations as represented in FIG. 6)
(SEQ ID NO: 18)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFHEVFNA
TTFASVYAWNRKRISNCVADYSVIYNFSPFFAFKCYGVSPTKLNDTCFTNVYADSFVIRGNE
TSQIAPGQTGNISDYNYKLPDDFTGCVIAWNSSKLDSKPSGNYNYLYRLFRKSKLKPFERDI
STEIYQAGNRSCNGVAGPNCTSPLQSYGFRPTYGVGHQPYRVVVLSFELLHAPATVCGPKKS
T
(Masked SARS-COV-2 XBB.1.16 RBD fused with LS i.e. SEQ ID NO: 14
with 7 specific mutations as represented in FIG. 7)
(SEQ ID NO: 23)
MQIYEGKLTAEGLRFGIVASRANHALVDRIVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFHEVFNA
TTFASVYAWNRKRISNCVADYSVIYNFSPFFAFKCYGVSPTKLNDTCFTNVYADSFVIRGNE
TSQIAPGQTGNISDYNYKLPDDFTGCVIAWNSSKLDSKPSGNYNYLYRLFRKSKLKPFERDI
STEIYQAGNRPCNGVAGPNCYSPLQSYGFRPTYGVGHQPYRVVVLSFELLHAPATVCGPKKS
T
(Masked SARS-COV-2 XBB.1.16 RBD fused with LS i.e. SEQ ID NO: 14
with 5 specific mutations)
(SEQ ID NO: 19)
MQIYEGKLTAEGLRFGIVASRANHALVDRLVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFHEVFNA
TTFASVYAWNRKRISNCVADYSVIYNFSPFFAFKCYGVSPTKLNDTCFTNVYADSFVIRGNE
TSQIAPGQTGNIADYNYKLPDDFTGCVIAWNSSKLDSKPSGNYNYLYRLFRKSKLKPFERDI
STEIYQAGNRSCNGVAGPNCTSPLQSYGERPTNGTGHQPYRVVVLSFELLHAPATVGPKKST
(Masked SARS-COV-2 XBB.1.16 RBD fused with LS i.e. SEQ ID NO: 14
with reverse mutation Y50IN and 7 specific mutations as represented in FIG. 8)
(SEQ ID NO: 24)
MQIYEGKLTAEGLRFGIVASRANHALVDRIVEGAIDAIVRHGGREEDITLVRVCGSWEIPVA
AGELARKEDIDAVIAIGVLCRGATPSFDYIASEVSKGLADLSLELRKPITFGVITADTLEQA
IEAAGTCHGNKGWEAALCAIEMANLFKSLRGGSGGSGGSGGSGGGRFPNITNLCPFHEVFNA
TTFASVYAWNRKRISNCVADYSVIYNFSPFFAFKCYGVSPTKLNDTCFTNVYADSFVIRGNE
TSQIAPGQTGNIADYNYKLPDDFTGCVIAWNSSKLDSKPSGNYNYLYRLFRKSKLKPFERDI
STEIYQAGNRPCNGVAGPNCYSPLQSYGFRPTNGTGHQPYRVVVLSFELLHAPATVGPKKST
(Masked SARS-COV-2 XBB.1.16 RBD fused with LS i.e. SEQ ID NO: 14
with reverse mutation Y501N and 5 specific mutations)

In another aspect, the disclosure provides multimers, comprising two or more copies of the isolated polypeptide of any embodiment or combination of embodiments disclosed herein. The multimers may be formed in any suitable manner, including but not limited to by inclusion of multimerization domains in the primary amino acid sequence, or by linking the polypeptides to a scaffold. In one embodiment, the multimer comprises between 2 and 60 copies of the isolated polypeptide. In various embodiments, the multimer may comprise 2, 3, 4, 6, 8, 60, or more copies of the polypeptide. In one embodiment, the disclosure provides scaffolds comprising two or more isolated polypeptides of any embodiment or combination of embodiments disclosed herein on a surface of the scaffold. Any suitable scaffolds may be used, whether polypeptide scaffolds, virus-like particles, beads, or other scaffold materials. The polypeptides may be linked to the scaffolds in any suitable matter. In one embodiment, the two or more isolated polypeptides are all identical polypeptides. In another embodiment, the two or more isolated polypeptides include different polypeptides, permitting delivery of a multivalent composition to a subject in need thereof.

In another aspect, the disclosure provides isolated nucleic acids encoding the isolated polypeptide of any embodiment or combination of embodiments disclosed herein. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit.

In a further aspect, the present disclosure provides host cells that comprise the polypeptides, nucleic acids, expression vectors and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In one embodiment of the nucleic acids of the disclosure, the nucleic acid comprises mRNA. Messenger RNA (mRNA) offers a relatively safe and efficient alternative to the polypeptide therapeutics and vaccines of the disclosure. After mRNA in vivo injection and uptake by professional antigen-presenting cells (APCs) in various tissues the multimerized RBD is expressed in APCs and displayed for the immune response, alternatively after mRNA uptake by cells in various tissues the polypeptides of the disclosure are expressed and secreted and the assembled multimerized RBD nanoparticle travels to lymph nodes to activate B cells and induce the classical pathway of complement activation, resulting in durable antibody responses.

Various modifications of mRNA may be used in order to counter the degradation of a RBD mRNA therapeutic or vaccine disclosed herein.

In one embodiment, the mRNA comprises a 5′ cap. The 5′ cap is a specially altered nucleotide on the 5′ end of mRNA. This process, known as mRNA capping, is highly regulated and vital in the creation of stable and mature messenger RNA able to undergo translation during protein synthesis. In eukaryotes the 5′ cap consists of a guanine nucleotide connected to mRNA via an unusual 5′ to 5′ triphosphate linkage. This guanosine is methylated directly after capping in vivo by a methyltransferase. In multicellular eukaryotes further modifications exist, including the methylation of the first 2 ribose sugars of the 5′ end of the mRNA. The 5′ cap is chemically similar to the 3′ end of an RNA molecule and this provides significant resistance to 5′exonucleases. Eukaryotic RNA undergoes a series of modifications in order to be exported from the nucleus and successfully translated into function proteins, many of which are dependent on mRNA capping, the first mRNA modification to take place. Various versions of 5′ caps can be added during or after the transcription reaction using various capping enzymes such as a vaccinia virus capping enzyme or by incorporating a synthetic cap or anti-reverse cap analogues.

In another embodiment, the mRNA further comprises a poly(A) tail of between 50 and 120 contiguous adenosine residues. Polyadenylation helps protect the mRNA 3′ end against degradation by exonucleases, the export of mature mRNA to the cytoplasmic environment, and also for mRNA translation.

In another embodiment, the mRNA comprises a 5′ untranslated region comprising the sequence GGGAGACUGCCACCAUG (SEQ ID NO: 29) or GGGAGACUGCCAAGAUG (SEQ ID NO: 30). The 5′-untranslated region (5′-UTR) of mRNA of this embodiment contains structural elements, which are recognized by cell-specific RNA-binding proteins, thereby affecting the translation of the molecule. To create recombinant RNA transcripts with short synthetic 5′-UTRs, the corresponding DNA sequences may be cloned into a plasmid vector upstream of the RBD gene. Table 1 lists the positions of different bases in the mRNA relative to the start codon. T7 promoter (TAATACGACTCACTATA (SEQ ID NO: 31)) may be combined with the Kozak element consensus sequence upstream of the start codon (ATG). Transcription from T7 promoter begins with the first G after the TATA element. The following six bases after the TATA element (GGGAGA) help provide high yields and homogenous 5′mRNA ends during in vitro transcription. This template-sequence results in an RNA, which has the sequence GGGAGACUGCCA(C/A) (C/G)AUG (SEQ ID NO: 32) as its 5′-UTR.

Table 1 shows two minimal UTRs with best results as 5′-UTRs.

TABLE 1
Sequences of Synthetic 5′-Untranslated Region

| Minimal | | Transcription | Extra | Kozak | |
|---|---|---|---|---|---|
| UTR | Promoter | start site | nucleotides | sequence | Start codon |
| UTR1 | T7 | GGGAGA | CT | GCCACC | ATG |
| UTR2 | T7 | GGGAGA | CT | GCCAAG | ATG |

In a further embodiment, the mRNA comprises a 3′ untranslated region comprising one or two copies of a beta globin mRNA 3′-UTR. Any beta globin mRNA 3′-UTR may be used as deemed suitable for an intended purpose. In one embodiment, the beta globin mRNA 3′-UTR comprises the amino acid sequence of SEQ ID NO: 26.

(SEQ ID NO: 26)
GCUCGCUUUCUUGCUGUCCAAUUUCUAUUAAAGGUUCCUUUGUUCCCUA
AGUCCAACUACUAAACUGGGGGAUAUUAUGAAGGGCCUUGAGCAUCUGG
AUUCUGCCUAAUAAAAAACAUUUAUUUUCAUUGC

In another aspect, the disclosure provides pharmaceutical compositions comprising
(a) the polypeptide, the multimer, the scaffold, the nucleic acid, the recombinant expression vector, and/or the cell of any embodiment or combination of embodiments disclosed herein; and
(b) a pharmaceutically acceptable carrier.

The pharmaceutical compositions of the disclosure may be used, for example, in the methods of the disclosure. In one embodiment the composition comprises the pharmaceutically acceptable carrier and:
(a) the isolated polypeptide of SEQ ID NO: 12; or
(b) the isolated polypeptide of SEQ ID NO: 13; or
(c) the isolated polypeptide of SEQ ID NO: 14; or
(d) the isolated polypeptide of SEQ ID NO: 17;
(e) the isolated polypeptide of SEQ ID NO: 18;
(f) the isolated polypeptide of SEQ ID NO: 19;
(g) the isolated polypeptide of SEQ ID NO: 23; or
(h) the isolated polypeptide of SEQ ID NO: 24.

In another embodiment, compositions comprise
(a) the mRNA of any embodiment or combination of embodiments herein; and
(b) a cationic lipid carrier, such as a liposome; or
(c) a polycationic protein, such as protamine.

In one such embodiment, the mRNA encodes:
(a) the isolated polypeptide of SEQ ID NO: 12; or
(b) the isolated polypeptide of SEQ ID NO: 13; or
(c) the isolated polypeptide of SEQ ID NO: 14; or
(d) the isolated polypeptide of SEQ ID NO: 17;
(e) the isolated polypeptide of SEQ ID NO: 18;
(f) the isolated polypeptide of SEQ ID NO: 19;
(g) the isolated polypeptide of SEQ ID NO: 23; or
(h) the isolated polypeptide of SEQ ID NO: 24.

The pharmaceutical composition may further comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptide, the multimer, the scaffold, the nucleic acid, the recombinant expression vector, and/or the cell of any embodiment or combination of embodiments herein may be the sole active agent in the pharmaceutical composition, or the composition may further comprise one or more other active agents suitable for an intended use.

In another aspect, the disclosure provides methods for treating a SARS coronavirus infection, comprising administering to a subject infected with a SARS coronavirus an amount effective to treat the infection of the polypeptide, the multimer, the scaffold, the nucleic acid, the recombinant expression vector, the cell, and/or the pharmaceutical composition of any embodiment or combination of embodiments disclosed herein.

As used herein, “treat” or “treating” means accomplishing one or more of the following in an individual that already has a SARS coronavirus infection: (a) reducing the severity of the infection; (b) limiting or preventing development of symptoms characteristic of the infection being treated; (c) inhibiting worsening of symptoms characteristic of the infection; and (d) limiting or preventing recurrence of symptoms in patients that were previously symptomatic for the infection.

In another aspect, the disclosure provides methods for limiting development of a SARS coronavirus infection, comprising administering to a subject at risk of SARS coronavirus infection an amount effective to limit development of a SARS coronavirus infection of the polypeptide, the multimer, the scaffold, the nucleic acid, the recombinant expression vector, the cell, and/or the pharmaceutical composition of any embodiment or combination of embodiments disclosed herein.

As used herein, “limiting” or “limiting development of” means accomplishing one or more of the following in an individual that does not have a SARS coronavirus infection: (a) preventing infection; (b) reducing the severity a subsequent infection; and (c) limiting or preventing development of symptoms after a subsequent infection.

In a further aspect, the disclosure provides methods for generating an immune response in a subject, comprising administering to the subject an amount effective to generate an immune response of the polypeptide, the multimer, the scaffold, the nucleic acid, the recombinant expression vector, the cell, and/or the pharmaceutical composition of any embodiment or combination of embodiments disclosed herein.

In this aspect, generating an immune response can be used to prevent infection, treat an existing infection or limit development of a subsequent infection.

In all of the above aspects, an “amount effective” refers to an amount of the therapeutic that is effective for treating and/or limiting the infection. The polypeptide, the multimer, the scaffold, the nucleic acid, the recombinant expression vector, the cell, and/or the pharmaceutical composition may be administered by any suitable route. In one embodiment of all of these aspect, the polypeptide, the multimer, the scaffold, the nucleic acid, the recombinant expression vector, the cell, and/or the pharmaceutical composition may be administered by subcutaneous, intradermal or intramuscular injection.

The subject in any of the methods disclosed herein may be any subject infected with or at risk or a SARS coronavirus infection, including but not limited to a human subject.

In another aspect, the disclosure provides methods for monitoring a SARS coronavirus-induced disease in a subject and/or monitoring response of the subject to immunization by a SARS coronavirus vaccine, comprising contacting the polypeptide, the multimer, the scaffold, the nucleic acid, the recombinant expression vector, the cell, and/or the pharmaceutical composition of any embodiment or combination of embodiments disclosed herein with a bodily fluid from the subject and detecting SARS coronavirus-binding antibodies in the bodily fluid of the subject. In this embodiment, a change in SARS coronavirus-binding antibodies in the bodily fluid of the subject can be monitored over time after the therapeutic or prophylactic methods disclosed herein, or any other therapeutic or prophylactic methods to treat or limit development of a SARS coronavirus-induced disease.

In one embodiment, the bodily fluid comprises serum or whole blood.

In a further aspect, the disclosure provides methods for detecting SARS coronavirus binding antibodies, comprising
(a) contacting the polypeptide, the multimer, the scaffold, and/or the pharmaceutical composition of any embodiment or combination of embodiments disclosed herein with a composition comprising a candidate SARS coronavirus binding antibody under conditions suitable for binding of SARS coronavirus antibodies to the polypeptide, the multimer, the scaffold, and/or the pharmaceutical composition; and
(b) detecting SARS coronavirus antibody complexes with the polypeptide, the multimer, the scaffold, and/or the pharmaceutical composition. In this embodiment, the reagents disclosed herein can be used in testing a subject for SARS coronavirus infection.

In one embodiment, the method further comprises isolating the SARS coronavirus antibodies that can be used, for example, as therapeutic antibodies to treat a subject having a SARS coronavirus infection.

In a further embodiment, the disclosure provides methods for producing SARS coronavirus antibodies, comprising
(a) administering to a subject an amount effective to generate an antibody response of the polypeptide, the multimer, the scaffold, the nucleic acid, the recombinant expression vector, the cell, and/or the pharmaceutical composition of any embodiment or combination of embodiments disclosed herein; and
(b) isolating antibodies produced by the subject. In this aspect, antibodies may be isolated and used, for example, as therapeutic antibodies to treat a subject having a SARS coronavirus infection.
   Example 1. Immunogenicity in Mouse of Omicron XBB.1.16 Masked RBD mRNA Vaccine VX3026R2 Encoding for Amino Acid Sequence of SEQ ID NO: 19 or mRNA Vaccine VX3026R2A Encoding for Amino Acid Sequence of SEQ ID NO: 24.

The following experiments confirmed that the mRNA vaccine VX3026R2 or VX3026R2A encoding for the amino acid sequence of SEQ ID NO: 19 or SEQ ID NO: 24 elicits class 5 neutralizing antibodies targeting a highly conserved epitope region of the RBD in vaccinated mouse.

Construct in pUC19 Plasmid

A construct with the 5′ minimal untranslated region UTR2 of Table 1, the human IL-2 signal sequence, a nucleotide sequence encoding the amino acid sequence of SEQ ID NO: 19 or SEQ ID NO: 24 and an N-terminal signal sequence (MYRMQLLSCIALSLALVINS; SEQ ID NO: 25), the 3′UTR region of SEQ ID NO: 26, a poly(A) tail of 80 adenosine residues and the BsmBI restriction site was cloned into the pUC19 vector.

mRNA Transcription and Capping

The supercoiled pUC19 DNA was upscaled, linearized with the enzyme BsmBI, and purified. In vitro transcription (IVT) was performed with T7 polymerase in a 5 mL reaction. The mRNA was capped with a cap 1 structure on the 5′ end by vaccinia 2′-O-methyltransferase enzymatic capping. Capped mRNA was purified by reverse phase chromatography followed by tangential flow filtration (TFF). Final yield of purified mRNA was 3.07 mg per mL of IVT reaction for both vaccines.

### Vaccine Formulation

The mRNA VX3026R2 or VX3026R2A was complexed with the polycationic protein protamine by addition of protamine to the mRNA at a mass ratio of 1:5. The VX3026R2 or VX3026R2A vaccine was prepared on each injection day with final VX3026R2 or VX3026R2A mRNA concentration of 840 μg/mL.

### Immunization

Two groups of N=10 CB6F1/J female mice 8 weeks old were dosed by intramuscular injection at the caudal thigh with a needle-free injection system (Tropis injector modified for mouse injection, PharmaJet) under 1-5% isoflurane anesthesia at week 0 and 3. Group 1 received the VX3026R2 vaccine and Group 2 the VX3026R2A vaccine. Dose was 42 μg/50 μL.

### Blood Collection

Blood was collected into clot activator tubes via retro-orbital capillary tube collection under 1-5% isoflurane anesthesia at 200 μL per collection in week 0 (prior to dose) and 3 (prior to second dose) and via cardiac puncture (500 μL per collection) in week 6. All blood samples were allowed to clot at room temperature, centrifuged ambient (20° C.) at 3000 RPM for 15 minutes, and serum supernatant was stored frozen at −80° C.

### ELISA Analysis

Sera samples of week 6 were analyzed with a SARS-CoV-2 anti-RBD antibody detection ELISA. The sandwich ELISA detects any antibodies directed to the wild-type RBD, including class 5 antibodies. The ELISA has four key components: a nickel chelate-coated 96-well plate (Pierce ThermoFisher, Cat #15442), a SARS-CoV-2 RBD protein (wild-type residues 319-541) produced in HEK293 cells with hexahistidine tag on the C-terminus used for plate capture (The Native Antigen, Cat #REC3182-100), a horseradish peroxidase (HRP) conjugated goat anti-mouse immunoglobulin G fragment crystallizable region (IgG Fc) secondary antibody (Pierce ThermoFisher, Cat #SA510276), and a mouse monoclonal anti-RBD antibody (DH6) (The Native Antigen, Cat #MAB12444-100) used as positive control.

First, the nickel chelate-coated plate was pre-washed. The plate was then coated with wild-type RBD protein by adding 0.5 μg of polyhistidine-tagged RBD per well and incubating with shaking for 1 hour at room temperature (20-25° C.). After washing steps, the positive control with a series of dilutions, the negative control (phosphate buffered saline, PBS) and the 20 sera samples were added in duplicate to the wells and incubated for 1 hour at room temperature. After washing steps antibodies in mouse sera targeting the wild-type RBD remained bound to the plate. The HRP-conjugated secondary anti-mouse IgG Fc antibody was added to detect the mouse antibodies on the plate and incubated for 1 hour at room temperature. After washing steps 3,3′,5,5′-tetramethylbenzidine (TMB) solution was added and the plate was incubated in dark at 20° C. for 15 min. The reaction was quenched by adding 0.16M sulfuric acid and the final solution was read immediately at 450 nm in a microtiter plate reader. The absorbance of the sample was dependent on the concentration of anti-RBD antibodies in the sample as indicated by the positive control anti-RBD monoclonal antibody. The positive control curve indicated a sensitivity of the assay of 1 ng/ml of antibody and optical density (OD) a linear function of antibody concentration below 10 ng/ml.

### Results

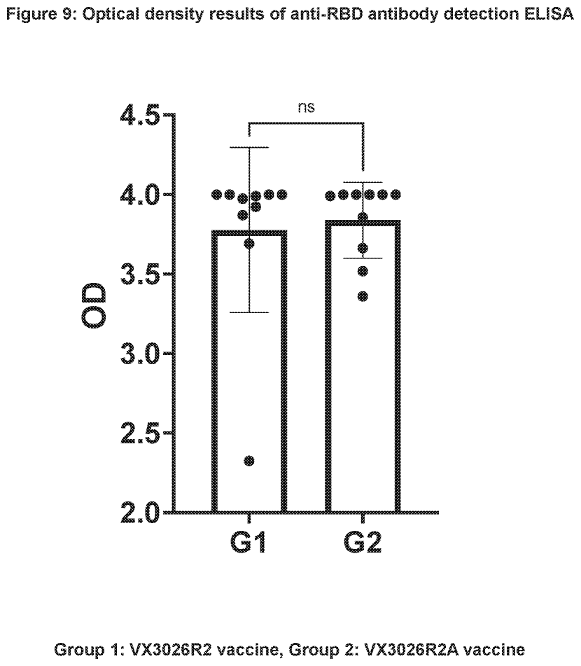

The sandwich ELISA detected any antibodies in week 6 mouse sera samples targeting the wild-type RBD, including class 5 antibodies, with a sensitivity of 1 ng/ml of antibody and optical density (OD) a linear function of antibody concentration below 10 ng/ml. In all 10 mice immunized with VX3026R2 and in all 10 mice immunized with VX3026R2A antibodies targeting the wild-type RBD were detected. In the group immunized with VX3026R2A a high concentration of antibodies (>10 ng/ml) was detected in all 10 mouse sera with OD above 3.36 (mean 3.84, sd 0.24), in the group immunized with VX3026R2 a high concentration of antibodies (>10 ng/ml) was detected in 9 out of 10 mouse sera with OD above 3.69 (mean 3.93, sd 0.10). The two vaccines were not significantly different (unpaired t test p-value=0.74) (FIG. 9).

Despite the long antigenic distance between the wild type and the XBB.1.16 backbone (21 mutations in the RBD) we found high serum antibody titers against the wild-type RBD in all week 6 mouse sera elicited by the two vaccines VX3026R2 and VX3026R2A. These results are consistent with epitope-focused design of the two vaccines with glycan masking of the variable regions of the RBD on the XBB.1.16 RBD backbone, and confirm that antibodies targeted to the unmasked conserved regions of the XBB.1.16 RBD, such as class 5 neutralizing antibodies, predominate in mouse sera elicited by the two vaccines. The conserved class 5 epitope region of the RBD is identical in the wild-type and the XBB.1.16 backbone except for two mutations (R346T and N460K). Specifically, for three class 5 neutralizing antibodies, the mutation N460K is located outside the epitopes of CC25.4, CC25.56, and CC25.43, and the mutation R346T is located outside the epitopes of CC25.4 and CC25.56, therefore these three class 5 neutralizing antibodies retain their affinity to the wild-type epitopes. Accordingly, we found high anti-wild-type RBD antibody titers in the sera of mice immunized by the two vaccines.

1. An isolated polypeptide comprising
(a) a SARS-CoV-2 receptor binding domain (RBD) comprising at least one non-naturally occurring N-linked glycosylation motif in any of the residues 369-372, 388-390, 405-407, 417-419, 437-441, 477-479, 487-489 and 501-503; and
(b) a multimerization domain.

2-24. (canceled)

25. A multimer comprising 60 or more copies of the polypeptide of claim 1.

26. (canceled)

27. A scaffold, comprising 60 or more isolated polypeptides of claim 1 on a surface of the scaffold.

28. (canceled)

29. A nucleic acid encoding the isolated polypeptide of claim 1.

30. A recombinant expression vector comprising the nucleic acid of claim 29 operatively linked to a suitable control sequence.

31. A recombinant host cell comprising the recombinant expression vector of claim 30.

32. The nucleic acid of claim 29 wherein the nucleic acid comprises mRNA.

33. The nucleic acid of claim 32, wherein the mRNA comprises a 5′ cap.

34. The nucleic acid of claim 32, further comprising a poly(A) tail of between 50 and 120 contiguous adenosine residues.

35. The nucleic acid of claim 32, wherein the mRNA comprises a 5′ untranslated region comprising the sequence GGGAGACUGCCACCAUG (SEQ ID NO: 29) or GGGAGACUGCCAAGAUG (SEQ ID NO: 30).

36. The nucleic acid of claim 32, wherein the mRNA comprises a 3′ untranslated region comprising one or two copies of a beta globin mRNA 3′-UTR.

37. The nucleic acid of claim 36, wherein the beta globin mRNA 3′-UTR comprises the amino acid sequence of SEQ ID NO: 26.

38. A pharmaceutical composition comprising
(a) the nucleic acid of claim 32; and
(b) a pharmaceutically acceptable carrier.

39. (canceled)

40. The pharmaceutical composition of claim 38, comprising:
a cationic lipid carrier, such as a liposome, or a polycationic protein, such as protamine.

41. The pharmaceutical composition of claim 40, wherein the mRNA encodes:
(a) the isolated polypeptide of SEQ ID NO: 12; or
(b) the isolated polypeptide of SEQ ID NO: 13; or
(c) the isolated polypeptide of SEQ ID NO: 14; or
(d) the isolated polypeptide of SEQ ID NO: 17; or
(e) the isolated polypeptide of SEQ ID NO: 18; or
(f) the isolated polypeptide of SEQ ID NO: 23; or
(g) the isolated polypeptide of SEQ ID NO: 19; or
(h) the isolated polypeptide of SEQ ID NO: 24.

42. A method for treating or limiting development of a SARS coronavirus infection, comprising administering to a subject infected with a SARS coronavirus an amount effective to treat the infection or limit development of the infection of the nucleic acid of claim 32.

43-45. (canceled)

46. A method for monitoring a SARS coronavirus-induced disease in a subject and/or monitoring response of the subject to immunization by a SARS coronavirus vaccine, comprising contacting the polypeptide of claim 1 with a bodily fluid from the subject and detecting SARS coronavirus-binding antibodies in the bodily fluid of the subject.

47. (canceled)

48. A method for:
(I) detecting SARS coronavirus binding antibodies, comprising
(a) contacting the polypeptide of claim 1; and
(b) detecting SARS coronavirus antibody complexes with the polypeptide; or
(II) producing SARS coronavirus antibodies, comprising
(a) administering to a subject amount effective to generate an antibody response of the polypeptide of claim 1, and
(b) isolating antibodies produced by the subject.

49-50. (canceled)


SEQUENCE LISTING (57 sequences)

SEQ ID NO: 1 - protein, 194 aa, synthetic construct
  VARIANT 9: note = X is G, D, or H
  VARIANT 16: note = X is R, K or T
  VARIANT 38: note = X is L or I
  VARIANT 39: note = X is Y or N
  VARIANT 41: note = X is S, F, or L
  VARIANT 42: note = X is A, S, or T
  VARIANT 43: note = X is S or P
  VARIANT 45: note = X is S or F
  VARIANT 46: note = X is T or A
  VARIANT 60: note = X is L, S, or T
  VARIANT 75: note = X is D or N
  VARIANT 77: note = X is V, S, or T
  VARIANT 78: note = X is R or S
  VARIANT 87: note = X is K, N, or T
  VARIANT 89: note = X is A, S, or T
  VARIANT 109: note = X is N, S, or T
  VARIANT 110: note = X is N or K
  VARIANT 111: note = X is L, S, or T
  VARIANT 115: note = X is V or P
  VARIANT 116: note = X is G or S
  VARIANT 122: note = X is L, Q, R, or M
  VARIANT 130: note = X is N or K
  VARIANT 147: note = X is S or N
  VARIANT 148: note = X is T, K, or R
  VARIANT 149: note = X is P, S, or T
  VARIANT 154: note = X is E, K, Q, or A
  VARIANT 156: note = X is F, P, S, or V
  VARIANT 159: note = X is Y, S, or T
  VARIANT 160: note = X is F or S
  VARIANT 163: note = X is Q or R
  VARIANT 166: note = X is G or S
  VARIANT 168: note = X is Q or R
  VARIANT 171: note = X is N or Y
  VARIANT 173: note = X is V, S, or T
  VARIANT 175: note = X is Y or H
NITNLCPFXEVFNATXFASVYAWNRKRISNCVADYSVXXNXXXFXXFKCYGVSPTKLNDXCFTNVYADSFVIRGXEXXQIAPGQTGXIXDYNYKLPDDFTGCVIAWNSXXXDSKXXGNYNYXYRLFRKSXLKPFERDISTEIYQAGXXXCNGVXGXNCXXPLXSYXFXPTXGXGXQPYRVVVLSFELLHAPATV

SEQ ID NO: 2 - protein, 194 aa, synthetic construct
NITNLCPFGEVFNATRFASVYAWNRKRISNCVADYSVLXNSXSFSTFKCYGVSPTKLNDXCFTNVYADSFVIRGDEVRQIAPGQTGKIADYNYKLPDDFTGCVIAWNSXNXDSKVGGNYNYLYRLFRKSNLKPFERDISTEIYQAGSTPCNGVEGFNCXFPLQSYGFQPTNGXGYQPYRVVVLSFELLHAPATV

SEQ ID NO: 3 - protein, 194 aa, synthetic construct
  VARIANT 42: note = X is A, S, or T
  VARIANT 60: note = X is L, S, or T
  VARIANT 77: note = X is V, S, or T
  VARIANT 89: note = X is A, S, or T
  VARIANT 109: note = X is N, S, or T
  VARIANT 111: note = X is L, S, or T
  VARIANT 149: note = X is P, S, or T
  VARIANT 159: note = X is Y, S, or T
NITNLCPFHEVFNATTFASVYAWNRKRISNCVADYSVIYNFXPFFAFKCYGVSPTKLNDXCFTNVYADSFVIRGNEXSQIAPGQTGNIXDYNYKLPDDFTGCVIAWNSXKXDSKPSGNYNYLYRLFRKSKLKPFERDISTEIYQAGNRXCNGVAGPNCXSPLQSYGFRPTYGVGHQPYRVVVLSFELLHAPATV

SEQ ID NO: 4 - protein, 194 aa, synthetic construct
  VARIANT 9: note = X is G, D, or H
  VARIANT 16: note = X is R, K, or T
  VARIANT 38: note = X is L or I
  VARIANT 41: note = X is S, F, or L
  VARIANT 43: note = X is S or P
  VARIANT 45: note = X is S or F
  VARIANT 46: note = X is T or A
  VARIANT 75: note = X is D or N
  VARIANT 78: note = X is R or S
  VARIANT 87: note = X is K, N, or T
  VARIANT 110: note = X is N or K
  VARIANT 115: note = X is V or P
  VARIANT 116: note = X is G or S
  VARIANT 122: note = X is L, Q, R, or M
  VARIANT 130: note = X is N or K
  VARIANT 147: note = X is S or N
  VARIANT 148: note = X is T, K, or R
  VARIANT 154: note = X is E, K, Q, or A
  VARIANT 156: note = X is F, P, S, or V
  VARIANT 160: note = X is F or S
  VARIANT 163: note = X is Q or R
  VARIANT 166: note = X is G or S
  VARIANT 166: note = X is Q or R
  VARIANT 171: note = X is N or Y
  VARIANT 175: note = X is Y or H
NITNLCPFXEVFNATXFASVYAWNRKRISNCVADYSVXYNXAXFXXFKCYGVSPTKLNDLCFTNVYADSFVIRGXEVXQIAPGQTGXIADYNYKLPDDFTGCVIAWNSNXLDSKXXGNYNYXYRLFRKSXLKPFERDISTEIYQAGXXPCNGVXGXNCYXPLXSYXFXPTXGVGXQPYRVVVLSFELLHAPATV

SEQ ID NO: 5 - protein, 194 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 6 - protein, 194 aa, synthetic construct
NITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATV

SEQ ID NO: 7 - protein, 154 aa, synthetic construct
  VARIANT 5: note = X is E or C
  VARIANT 8: note = X is L or C
  VARIANT 9: note = X is T or G
  VARIANT 22: note = X is F or A
  VARIANT 40: note = X is R or C
  VARIANT 41: note = X is H, F, or M
  VARIANT 52: note = X is R or C
  VARIANT 54: note = X is P or C
  VARIANT 61: note = X is V or C
  VARIANT 82: note = X is I or C
  VARIANT 86: note = X is T or C
  VARIANT 87: note = X is P or G
  VARIANT 88: note = X is H or S
  VARIANT 105: note = X is L or C
  VARIANT 119: note = X is D or C
  VARIANT 127: note = X is R or A
  VARIANT 131: note = X is K or C
  VARIANT 142: note = X is S or C
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLR

SEQ ID NO: 8 - protein, 154 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 9 - protein, 354 aa, synthetic construct
  VARIANT 5: note = X is E or C
  VARIANT 8: note = X is L or C
  VARIANT 9: note = X is T or G
  VARIANT 22: note = X is F or A
  VARIANT 40: note = X is R or C
  VARIANT 41: note = X is H, F, or M
  VARIANT 52: note = X is R or C
  VARIANT 54: note = X is P or C
  VARIANT 61: note = X is V or C
  VARIANT 82: note = X is I or C
  VARIANT 86: note = X is T or C
  VARIANT 87: note = X is P or G
  VARIANT 88: note = X is H or S
  VARIANT 105: note = X is L or C
  VARIANT 119: note = X is D or C
  VARIANT 127: note = X is R or A
  VARIANT 131: note = X is K or C
  VARIANT 142: note = X is S or C
  REGION 155..157: note = can be repeated n times
  VARIANT 169: note = X is G, D, or H
  VARIANT 176: note = X is R, K, or T
  VARIANT 198: note = X is L or I
  VARIANT 199: note = X is Y or N
  VARIANT 201: note = X is S, F, or L
  VARIANT 202: note = X is A, S, or T
  VARIANT 203: note = X is S or P
  VARIANT 205: note = X is S or F
  VARIANT 206: note = X is T or A
  VARIANT 220: note = X is L, S, or T
  VARIANT 235: note = X is D or N
  VARIANT 237: note = X is V, S, or T
  VARIANT 238: note = X is R or S
  VARIANT 247: note = X is K, N, or T
  VARIANT 249: note = X is A, S, or T
  VARIANT 269: note = X is N, S, or T
  VARIANT 270: note = X is N or K
  VARIANT 271: note = X is L, S, or T
  VARIANT 275: note = X is V or P
  VARIANT 276: note = X is G or S
  VARIANT 282: note = X is L,Q, R, or M
  VARIANT 290: note = X is N or K
  VARIANT 307: note = X is S or N
  VARIANT 308: note = X is T, K, or R
  VARIANT 309: note = X is P, S, or T
  VARIANT 314: note = X is E, K, Q, or A
  VARIANT 316: note = X is F, P, S, or V
  VARIANT 319: note = X is Y, S, or T
  VARIANT 320: note = X is F or S
  VARIANT 323: note = X is Q or R
  VARIANT 326: note = X is G or S
  VARIANT 328: note = X is Q or R
  VARIANT 331: note = X is N or Y
  VARIANT 333: note = X is V, S, or T
  VARIANT 335: note = X is Y or H
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFXEVFNATXFASVYAWNRKRISNCVADYSVXXNXXXFXXFKCYGVSPTKLNDXCFTNVYADSFVIRGXEXXQIAPGQTGXIXDYNYKLPDDFTGCVIAWNSXXXDSKXXGNYNYXYRLFRKSXLKPFERDISTEIYQAGXXXCNGVXGXNCXXPLXSYXFXPTXGXGXQPYRVVVLSFELLHAPATV

SEQ ID NO: 10 - protein, 354 aa, synthetic construct
  VARIANT 5: note = X is E or C
  VARIANT 8: note = X is L or C
  VARIANT 9: note = X is T or G
  VARIANT 22: note = X is F or A
  VARIANT 40: note = X is R or C
  VARIANT 41: note = X is H, F, or M
  VARIANT 52: note = X is R or C
  VARIANT 54: note = X is P or C
  VARIANT 61: note = X is V or C
  VARIANT 82: note = X is I or C
  VARIANT 86: note = X is T or C
  VARIANT 87: note = X is P or G
  VARIANT 88: note = X is H or S
  VARIANT 105: note = X is L or C
  VARIANT 119: note = X is D or C
  VARIANT 127: note = X is R or A
  VARIANT 131: note = X is K or C
  VARIANT 142: note = X is S or C
  REGION 155..157: note = CAN BY REPEATED N TIMES
  VARIANT 199: note = X is Y or N
  VARIANT 202: note = X is A, S, or T
  VARIANT 220: note = X is L, S, or T
  VARIANT 269: note = X is N, S, or T
  VARIANT 271: note = X is L, S, or T
  VARIANT 319: note = X is Y, S, or T
  VARIANT 333: note = X is V, S, or T
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGSSGGGNITNLCPFGEVFNATRFASVYAWNRKRISNCVADYSVLXNSXSFSTFKCYGVSPTKLNDXCFTNVYADSFVIRGDEVRQIAPGQTGKIADYNYKLPDDFTGCVIAWNSXNXDSKVGGNYNYLYRLFRKSNLKPFERDISTEIYQAGSTPCNGVEGFNCXFPLQSYGFQPTNGXGYQPYRVVVLSFELLHAPATV

SEQ ID NO: 11 - protein, 354 aa, synthetic construct
  VARIANT 5: note = X is E or C
  VARIANT 9: note = X is T or G
  VARIANT 8: note = X is L or C
  VARIANT 22: note = X is F or A
  VARIANT 40: note = X is R or C
  VARIANT 41: note = X is H, F, or M
  VARIANT 52: note = X is R or C
  VARIANT 54: note = X is P or C
  VARIANT 61: note = X is V or C
  VARIANT 82: note = X is I or C
  VARIANT 86: note = X is T or C
  VARIANT 87: note = X is P or G
  VARIANT 88: note = X is H or S
  VARIANT 105: note = X is L or C
  VARIANT 119: note = X is D or C
  VARIANT 127: note = X is R or A
  VARIANT 131: note = X is K or C
  VARIANT 142: note = X is S or C
  VARIANT 202: note = X is A, S, or T
  REGION 155..157: note = CAN BE REPEATED N TIMES
  VARIANT 220: note = X is L, S, or T
  VARIANT 237: note = X is V, S, or T
  VARIANT 249: note = X is A, S, or T
  VARIANT 269: note = X is N, S, or T
  VARIANT 271: note = X is L, S, or T
  VARIANT 309: note = X is P, S, or T
  VARIANT 319: note = X is Y, S, or T
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFHEVFNATTFASVYAWNRKRISNCVADYSVIYNFXPFFAFKCYGVSPTKLNDXCFTNVYADSFVIRGNEXSQIAPGQTGNIXDYNYKLPDDFTGCVIAWNSXKXDSKPSGNYNYLYRLFRKSKLKPFERDISTEIYQAGNRXCNGVAGPNCXSPLQSYGFRPTYGVGHQPYRVVVLSFELLHAPATV

SEQ ID NO: 12 - protein, 373 aa, synthetic construct
  VARIANT 181: note = X is G, D or H
  VARIANT 188: note = X is R, K, or T
  VARIANT 210: note = X is L or I
  VARIANT 211: note = X is Y or N
  VARIANT 213: note = X is S, F, or L
  VARIANT 214: note = X is A, S, or T
  VARIANT 215: note = X is S or P
  VARIANT 217: note = X is S or F
  VARIANT 218: note = X is T or A
  VARIANT 232: note = X is L, S, or T
  VARIANT 247: note = X is D or N
  VARIANT 249: note = X is V, S, or T
  VARIANT 250: note = X is R or S
  VARIANT 259: note = X is K, N, or T
  VARIANT 261: note = X is A, S, or T
  VARIANT 281: note = X is N, S, or T
  VARIANT 282: note = X is N or K
  VARIANT 283: note = X is L, S, or T
  VARIANT 287: note = X is V or P
  VARIANT 288: note = X is G or S
  VARIANT 294: note = X is L, Q, R, or M
  VARIANT 302: note = X is N or K
  VARIANT 319: note = X is S or N
  VARIANT 320: note = X is T, K, or R
  VARIANT 321: note = X is P, S, or T
  VARIANT 326: note = X is E, K, Q, or A
  VARIANT 328: note = X is F, P, S, or V
  VARIANT 331: note = X is Y, S, or T
  VARIANT 332: note = X is F or S
  VARIANT 335: note = X is Q or R
  VARIANT 338: note = X is G or S
  VARIANT 340: note = X is Q or R
  VARIANT 343: note = X is N or Y
  VARIANT 345: note = X is V, S, or T
  VARIANT 347: note = X is Y or H
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFXEVFNATXFASVYAWNRKRISNCVADYSVXXNXXXFXXFKCYGVSPTKLNDXCFTNVYADSFVIRGXEXXQIAPGQTGXIXDYNYKLPDDFTGCVIAWNSXXXDSKXXGNYNYXYRLFRKSXLKPFERDISTEIYQAGXXXCNGVXGXNCXXPLXSYXFXPTXGXGXQPYRVVVLSFELLHAPATVCGPKKST

SEQ ID NO: 13 - protein, 373 aa, synthetic construct
  VARIANT 211: note = X is Y or N
  VARIANT 214: note = X is A, S, or T
  VARIANT 232: note = X is L, S, or T
  VARIANT 281: note = X is N, S, or T
  VARIANT 283: note = X is L, S, or T
  VARIANT 331: note = X is Y, S, or T
  VARIANT 345: note = X is V, S, or T
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLXNSXSFSTFKCYGVSPTKLNDXCFTNVYADSFVIRGDEVRQIAPGQTGKIADYNYKLPDDFTGCVIAWNSXNXDSKVGGNYNYLYRLFRKSNLKPFERDISTEIYQAGSTPCNGVEGFNCXFPLQSYGFQPTNGXGYQPYRVVVLSFELLHAPATVCGPKKST

SEQ ID NO: 14 - protein, 373 aa, synthetic construct
  VARIANT 214: note = X is A, S, or T
  VARIANT 232: note = X is L, S, or T
  VARIANT 249: note = X is V, S, or T
  VARIANT 261: note = X is A, S, or T
  VARIANT 281: note = X is N, S, or T
  VARIANT 283: note = X is L, S, or T
  VARIANT 321: note = X is P, S, or T
  VARIANT 331: note = X is Y, S, or T
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFHEVFNATTFASVYAWNRKRISNCVADYSVIYNFXPFFAFKCYGVSPTKLNDXCFTNVYADSFVIRGNEXSQIAPGQTGNIXDYNYKLPDDFTGCVIAWNSXKXDSKPSGNYNYLYRLFRKSKLKPFERDISTEIYQAGNRXCNGVAGPNCXSPLQSYGFRPTYGVGHQPYRVVVLSFELLHAPATVCGPKKST

SEQ ID NO: 15-16 - skipped (2 entries)

SEQ ID NO: 17 - protein, 373 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFGEVFNATRFASVYAWNRKRISNCVADYSVLYNSSSFSTFKCYGVSPTKLNDTCFTNVYADSFVIRGDEVRQIAPGQTGKIADYNYKLPDDFTGCVIAWNSSNLDSKVGGNYNYLYRLFRKSNLKPFERDISTEIYQAGSTPCNGVEGFNCTFPLQSYGFQPTNGTGYQPYRVVVLSFELLHAPATVCGPKKST

SEQ ID NO: 18 - protein, 373 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFHEVFNATTFASVYAWNRKRISNCVADYSVIYNFSPFFAFKCYGVSPTKLNDTCFTNVYADSFVIRGNETSQIAPGQTGNISDYNYKLPDDFTGCVIAWNSSKLDSKPSGNYNYLYRLFRKSKLKPFERDISTEIYQAGNRSCNGVAGPNCTSPLQSYGFRPTYGVGHQPYRVVVLSFELLHAPATVCGPKKST

SEQ ID NO: 19 - protein, 372 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFHEVFNATTFASVYAWNRKRISNCVADYSVIYNFSPFFAFKCYGVSPTKLNDTCFTNVYADSFVIRGNETSQIAPGQTGNIADYNYKLPDDFTGCVIAWNSSKLDSKPSGNYNYLYRLFRKSKLKPFERDISTEIYQAGNRSCNGVAGPNCTSPLQSYGFRPTNGTGHQPYRVVVLSFELLHAPATVGPKKST

SEQ ID NO: 20 - protein, 194 aa, synthetic construct; identical to SEQ ID NO: 1
  VARIANT 9: note = X is G, D, or H
  VARIANT 16: note = X is R, K, or T
  VARIANT 38: note = X is L or I
  VARIANT 39: note = X is Y or N
  VARIANT 41: note = X is S, F, or L
  VARIANT 42: note = X is A or S
  VARIANT 43: note = X is S or P
  VARIANT 45: note = X is S or F
  VARIANT 46: note = X is T or A
  VARIANT 60: note = X is L or T
  VARIANT 75: note = X is D or N
  VARIANT 77: note = X is V or T
  VARIANT 78: note = X is R or S
  VARIANT 87: note = X is K, N, or T
  VARIANT 89: note = X is A or S
  VARIANT 109: note = X is N or S
  VARIANT 110: note = X is N or K
  VARIANT 111: note = X is L or T
  VARIANT 115: note = X is V or P
  VARIANT 116: note = X is G or S
  VARIANT 122: note = X is L, Q, R, or M
  VARIANT 130: note = X is N or K
  VARIANT 147: note = X is S or N
  VARIANT 148: note = X is T, K, or R
  VARIANT 149: note = X is P or S
  VARIANT 154: note = X is E, A, Q, or A
  VARIANT 156: note = X is F, P, S, or V
  VARIANT 159: note = X is Y or T
  VARIANT 160: note = X is F or S
  VARIANT 163: note = X is Q or R
  VARIANT 166: note = X is G or S
  VARIANT 168: note = X is Q or R
  VARIANT 171: note = X is N or Y
  VARIANT 173: note = X is V or T
  VARIANT 175: note = X is Y or H

SEQ ID NO: 21 - protein, 194 aa, synthetic construct; identical to SEQ ID NO: 2
  VARIANT 39: note = X is Y or N
  VARIANT 42: note = X is A or S
  VARIANT 60: note = X is L or T
  VARIANT 109: note = X is N or S
  VARIANT 111: note = X is L or T
  VARIANT 159: note = X is Y or T
  VARIANT 173: note = X is V or T

SEQ ID NO: 22 - protein, 194 aa, synthetic construct; identical to SEQ ID NO: 3
  VARIANT 42: note = X is A or S
  VARIANT 60: note = X is L or T
  VARIANT 77: note = X is V or T
  VARIANT 89: note = X is A or S
  VARIANT 109: note = X is N or S
  VARIANT 111: note = X is L or T
  VARIANT 149: note = X is P or S
  VARIANT 159: note = X is Y or T

SEQ ID NO: 23 - protein, 373 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFHEVFNATTFASVYAWNRKRISNCVADYSVIYNFSPFFAFKCYGVSPTKLNDTCFTNVYADSFVIRGNETSQIAPGQTGNISDYNYKLPDDFTGCVIAWNSSKLDSKPSGNYNYLYRLFRKSKLKPFERDISTEIYQAGNRPCNGVAGPNCYSPLQSYGFRPTYGVGHQPYRVVVLSFELLHAPATVCGPKKST

SEQ ID NO: 24 - protein, 372 aa, synthetic construct
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRFPNITNLCPFHEVFNATTFASVYAWNRKRISNCVADYSVIYNFSPFFAFKCYGVSPTKLNDTCFTNVYADSFVIRGNETSQIAPGQTGNIADYNYKLPDDFTGCVIAWNSSKLDSKPSGNYNYLYRLFRKSKLKPFERDISTEIYQAGNRPCNGVAGPNCYSPLQSYGFRPTNGTGHQPYRVVVLSFELLHAPATVGPKKST

SEQ ID NO: 25 - protein, 20 aa, synthetic construct
MYRMQLLSCIALSLALVTNS

SEQ ID NO: 26 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 27 - protein, 33 aa, synthetic construct
RMKQIEDKIEEILSKIYHIENEIARIKKLIGER

SEQ ID NO: 28 - protein, 34 aa, synthetic construct
MKVKQLEDVVEELLSVNYHLENVVARLKKLVGER

SEQ ID NO: 29 - RNA, 17 nt, synthetic construct
  source 1..17: mol_type = other RNA
gggagactgccaccatg

SEQ ID NO: 30 - RNA, 17 nt, synthetic construct
  source 1..17: mol_type = other RNA
gggagactgccaagatg

SEQ ID NO: 31 - DNA, 17 bp, synthetic construct
  source 1..17: mol_type = other DNA
taatacgactcactata

SEQ ID NO: 32 - RNA, 17 nt, synthetic construct
  source 1..17: mol_type = other RNA
  variation 13: note = n is c or a
  variation 14: note = n is c or g
gggagactgccannatg

SEQ ID NO: 33 - protein, 6 aa, synthetic construct
  VAR_SEQ 1..3: note = can be repeated
GGSGGG

SEQ ID NO: 34 - protein, 13 aa, synthetic construct
  VARIANT 9: note = X is G, D, or H
NITNLCPFXEVFN

SEQ ID NO: 35 - protein, 17 aa, synthetic construct
  VARIANT 3: note = X is R, K, or T
ATXFASVYAWNRKRISN

SEQ ID NO: 36 - protein, 32 aa, synthetic construct
  VARIANT 8: note = X is L or I
  VARIANT 11: note = X is S, F, or L
  VARIANT 15: note = X is S or F
  VARIANT 16: note = X is T or A
  VARIANT 13: note = X is S or P
CVADYSVXYNXAXFXXFKCYGVSPTKLNDLCF

SEQ ID NO: 37 - protein, 7 aa, synthetic construct
TNVYADS

SEQ ID NO: 38 - protein, 26 aa, synthetic construct
  VARIANT 6: note = X is D or N
  VARIANT 9: note = X is R or S
  VARIANT 18: note = X is K, N, or T
FVIRGXEVXQIAPGQTGXIADYNYKL

SEQ ID NO: 39 - protein, 5 aa, synthetic construct
PDDFT

SEQ ID NO: 40 - protein, 26 aa, synthetic construct
  VARIANT 10: note = X is N or K
  VARIANT 15: note = X is V or P
  VARIANT 16: note = X is G or S
  VARIANT 22: note = X is L, Q, R, or M
GCVIAWNSNXLDSKXXGNYNYXYRLF

SEQ ID NO: 41 - protein, 18 aa, synthetic construct
  VARIANT 4: note = X is N or K
RKSXLKPFERDISTEIYQ

SEQ ID NO: 42 - protein, 6 aa, synthetic construct
  VARIANT 3: note = X is S or N
  VARIANT 4: note = X is T, K, or R
AGXXPC

SEQ ID NO: 43 - protein, 9 aa, synthetic construct
  VARIANT 2: note = X is E, K, Q, or A
  VARIANT 4: note = X is F, P, S, or V
  VARIANT 8: note = X is F or S
VXGXNCYXP

SEQ ID NO: 44 - protein, 21 aa, synthetic construct
  VARIANT 1: note = X is Q or R
  VARIANT 4: note = X is G or S
  VARIANT 6: note = X is Q or R
  VARIANT 9: note = X is N or Y
  VARIANT 13: note = X is Y or H
XSYXFXPTXGVGXQPYRVVVL

SEQ ID NO: 45 - protein, 11 aa, synthetic construct
SFELLHAPATV

SEQ ID NO: 46 - protein, 32 aa, synthetic construct; identical to SEQ ID NO: 36
  VARIANT 8: note = X is L or I
  VARIANT 11: note = X is S, F, or L
  VARIANT 13: note = X is S or P
  VARIANT 15: note = X is S or F
  VARIANT 16: note = X is T or A

SEQ ID NO: 47 - protein, 32 aa, synthetic construct
  VARIANT 8: note = X is L or I
  VARIANT 11: note = X is S, F, or L
  VARIANT 12: note = X is S or T
  VARIANT 13: note = X is S or P
  VARIANT 15: note = X is S or F
  VARIANT 16: note = X is T or A
  VARIANT 30: note = X is S or T
CVADYSVXNNXXXFXXFKCYGVSPTKLNDXCF

SEQ ID NO: 48 - protein, 26 aa, synthetic construct
  VARIANT 6: note = X is D or N
  VARIANT 9: note = X is R or S
  VARIANT 18: note = X is K, N, or T
FVIRGXEVXQIAPGQTGXIADYNYKL

SEQ ID NO: 49 - protein, 26 aa, synthetic construct
  VARIANT 6: note = X is D or N
  VARIANT 8: note = X is S or T
  VARIANT 9: note = X is R or S
  VARIANT 18: note = X is K, N, or T
  VARIANT 20: note = X is S or T
FVIRGXEXXQIAPGQTGXIXDYNYKL

SEQ ID NO: 50 - protein, 26 aa, synthetic construct
  VARIANT 10: note = X is N or K
  VARIANT 15: note = X is V or P
  VARIANT 16: note = X is G or S
  VARIANT 22: note = X is L, Q, R, or M
GCVIAWNSNXLDSKXXGNYNYXYRLF

SEQ ID NO: 51 - protein, 26 aa, synthetic construct
  VARIANT 9: note = X is S or T
  VARIANT 10: note = X is N or K
  VARIANT 11: note = X is S or T
  VARIANT 15: note = X is V or P
  VARIANT 16: note = X is G or S
  VARIANT 22: note = X is L, Q, R, or M
GCVIAWNSXXXDSKXXGNYNYXYRLF

SEQ ID NO: 52 - protein, 6 aa, synthetic construct
  VARIANT 3: note = X is S or N
  VARIANT 4: note = X is T, K, or R
AGXXPC

SEQ ID NO: 53 - protein, 6 aa, synthetic construct
  VARIANT 3: note = X is S or N
  VARIANT 4: note = X is T, K, or R
  VARIANT 5: note = X is S or T
AGXXSC

SEQ ID NO: 54 - protein, 9 aa, synthetic construct
  VARIANT 2: note = X is E, K, Q, or A
  VARIANT 4: note = X is F, P, S, or V
  VARIANT 8: note = X is F or S
VXGXNCYXP

SEQ ID NO: 55 - protein, 9 aa, synthetic construct
  VARIANT 2: note = X is E, K, Q, or A
  VARIANT 4: note = X is F, P, S, or V
  VARIANT 7: note = X is S or T
  VARIANT 8: note = X is F or S
VXGXNCXXP

SEQ ID NO: 56 - protein, 21 aa, synthetic construct
  VARIANT 1: note = X is Q or R
  VARIANT 4: note = X is G or S
  VARIANT 6: note = X is Q or R
  VARIANT 9: note = X is N or Y
  VARIANT 13: note = X is Y or H
XSYXFXPTXGVGXQPYRVVVL

SEQ ID NO: 57 - protein, 21 aa, synthetic construct
  VARIANT 1: note = X is Q or R
  VARIANT 4: note = X is G or S
  VARIANT 6: note = X is Q or R
  VARIANT 9: note = X is N or Y
  VARIANT 11: note = X is S or T
  VARIANT 13: note = X is Y or H
XSYXFXPTXGXGXQPYRVVVL